(19) United States
(12) Patent Application Publication
(10) Pub. No.: US 2005/0014151 A1
(43) Pub. Date: Jan. 20, 2005

(54) Device with chemical surface patterns

(76) Inventors: Marcus Textor, Schaffhausen (CH); Roger MIchel, Muhlou (CH); Janos Voros, Schlieren (CH); Jeffrey A. Hubbell, Morges (CH); Jost Lussi, Zurich (CH)

Correspondence Address: BACHMAN & LAPOINTE, P.C., 900 CHAPEL STREET, SUITE 1201, NEW HAVEN, CT, 06510, US

(21) Appl. No.: 10/489,688

(22) PCT Filed: Sep. 12, 2001
(86) PCT No.: PCT/CH01/00548

(51) Int. Cl. C12Q 1/68; C12M 1/34

(52) U.S. Cl. 435/6; 435/287.2

(57) ABSTRACT

A device with chemical surface patterns (defined surface areas of at least two different chemical compositions) with biochemical or biological relevance on substrates with prefabricated patterns of at least two different types of regions (α, β, . . . ), whereas at least two different, consecutively applied molecular self-assembly systems (A, B, . . . ) are used in a way that at least one of the applied assembly systems (A or B or . . . ) is specific to one type of the prefabricated patterns (α or β or . . . ).

### BACKGROUND OF THE INVENTION

The invention relates to a device with chemical surface patterns, a bioanalytical sensing platform comprising the device, a method for the simultaneous determination of analytes and a biomedical device.

Chemical patterning of surfaces i.e., the generation of structures of different chemical composition on surfaces, either in a regular, geometric array or with a statistical distribution of features, is an important technique in a variety of application including microfabrication, microelectronics, micromechanics, biomaterials and biosensors [Kane, R. S., Takayama, S., Ostuni, E., Ingber, D. E., Whitesides, G. M., Patterning proteins and cells using soft lithography, Biomaterials 20 (1999) 2363-2376. Xia, Y., Rogers, J. A., Paul, K. E., Whitesides, G. M., Unconventional Methods for Fabricating and Patterning Nanostructures. Chem. Rev. 99 (1999), 1823-1848]. FIG. 1 shows examples of chemical patterns, which may or may not be connected with topographical variations (height differences) across the surface.

A large variety of techniques has been developed and described in the literature and in patents to produce patterns with more or less controlled chemical composition and structure in different areas of a surface. Examples include:

Type A: Techniques that involve the use of photoresists and/or etching procedures [Xia, Y., Rogers, J. A., Paul, K. E., Whitesides, G. M., Unconventional Methods for Fabricating and Patterning Nanostructures. Chem. Rev. 99 (1999), 1823-1848]
Lithography using visible, UV or X-ray exposure of photosensitive coatings (photoresists) through appropriate masks
Electron beam lithography
Writing structures by fast ion bombardement
Laser microstructuring
And many other techniques

Type B: Techniques that rely on self-assembly: A number of techniques use molecular self-assembly in combination with structuring techniques:
Microfluidic patterning (μFP) of surfaces in contact with stamps having channels that can be filled with a solution containing molecules that assemble on the exposed surface within the channels [Kane, R. S., Takayama, S., Ostuni, E., Ingber, D. E., Whitesides, G. M., Patterning proteins and cells using soft lithography, Biomaterials 20 (1999) 2363-2376].
Microcontact printing (μCP), where stamps with a particular structure are used to transfer material locally adsorbed at or absorbed in the stamp to the surface in a selective way [Kane, R. S., Takayama, S., Ostuni, E., Ingber, D. E., Whitesides, G. M., Patterning proteins and cells using soft lithography, Biomaterials 20 (1999) 2363-2376. Chiu, D. T., Jeon, N. L., Huang, S., Kane, R., Wargo, C. J., Choi, I. S., Ingber, D. E., Whitesides, G. M., Patterned deposition of cells and proteins onto surfaces by using three-dimensional microfluidic systems. Proc. Natl. Ac. Sci. 97 (2000), 2408-2413].
UV patterning of alkane thiols on gold surfaces through appropriate masks, resulting in spatially controlled oxidation of the thiol headgroup to sulfuroxide or sulfon, washing off the surface the less strongly bound oxidized alkane thiols and backfilling the unprotected gold areas with a different thiol [Xia Y N, Zhao X M, Whitesides G M, Pattern transfer: Self-assembled monolayers as ultrathin resists, Microelectronic Engineering 32 (1-4): 255-268, September 1996].

These standard techniques described above have, however, specific disadvantages:

Type A techniques, although partly suitable for mass scale production, in general only allow the fabrication of structures with relatively simple surface chemistries, meaning chemical compositions that have to be stable in the development stages of the lithographic process. In the biomaterial and biosensor area, however, there is a requirement to structure surfaces based on rather delicate, often labile molecules such as proteins, antibodies or nucleic acids (DNA or RNA). The harsh conditions of the lithographic fabrication steps are likely to be incompatible with these types of biochemical or biological structures.

Type B: While these techniques allow the spatially-controlled transfer of highly sensitive molecules such as proteins, they always involve a local contact of the surface with the stamping material, which may lead to the transfer of unwanted stamp material and thus local contamination that may interfere with the functionality of the surface. The standard stamp materials are based on elastomeric siloxane or silicon type of polymers [Patent number: WO 9629629, publication date: Sep. 26, 1996, inventor(s): Jackman Rebecca J, Whitesides George M; Biebuyck Hans; Kim Enoch; Mrksich Milan; Berggren Karl K; Gorman Chris; Kumar Amit; Prentiss Mara G; Wilbur James L; Xia Younan, Applicant(s): Harvard College (US)], such as polydimethylsiloxane, and these are particularly critical in terms of transfer of low-molecular-weight or monomeric components of the stamp elastomer to surfaces, leading to hydrophobic contaminated contact surfaces, which are likely to interfere with subsequent modification procedures. Another major disadvantage is the lack of reproducibility due to variations in quality from stamp to stamp, and the general difficulty of patterning large areas due to difficulties of achieving a perfect stamp-surface contact area over larger dimensions. Moreover, there are restrictions in the type of patterns that can be produced by stamping using elastomer stamps; e.g. widely-spaced patterns can not be transferred efficiently due to the sagging of the stamp. Finally, when using stamps in production, there is a continuous deterioration in the fidelity of the stamping process over the life time of a stamp.

The invention aims at eliminating some of the major disadvantages and limitations of the known techniques described in the introduction. Firstly, it aims at providing a patterning technology that allows one to pattern large surfaces and large batch sizes in a very reproducible way, with almost no limitations in terms of the geometry and dimensions of the patterns. Secondly, it provides the ability to fabricate patterns with biochemical or biological structures such as peptides, proteins, or nucleci acids. A particularly important aim is to pattern surfaces into areas that are resistant to interactions with biological media, meaning, in particular, resistance to the adsorption of biomolecules (e.g. proteins, carbohydrates or nucleic acids) and cells, and areas that elicit specific biological responses, such as antibody-antigen interactions or cell receptor-surface interactions.

There are a number of particular needs that the invented technique is able to address:
Flexibility-basically without limitations-with respect to the geometry and size of the features, ranging from the mm across the micrometer to the submicrometer and nanometer range.
Stringent control over the physico-chemical properties of the pattern areas.
Extremely high contrast between adhesive and non-adhesive areas, meaning very high ratios of protein adsorption or cell attachment on the adhesive area in relation to the non-adhesive “background”.
The possibility that the biochemical or biological modification is directly linked to the selective adsorption in areas of defined physico-chemical properties.
Stringent control over the density, conformation, orientation and therefore functionality of biochemically or biologically active sites immobilized in specific areas of the pattern.
High reproducibility and fidelity of the pattern chemistry and biology across large surfaces areas, and with little or no variations from batch to batch.

### SUMMARY OF THE INVENTION

The objectives are met by a device with a chemical surface patterns (defined surface areas of at least two different chemical compositions) with biochemical or biological relevance on substrates with prefabricated patterns (“prepatterns”) of at least two different types of regions (called α, β, . . . ), whereas at least two different, consecutively applied molecular self-assembly systems (called A, B, . . . ) are used in a way that at least one of the applied assembly systems (A or B or . . . ) is specific to one type of the prefabricated patterns (α or β or . . . ) (see schematic process scheme in FIG. 2).

A preferred example is a device where the specificity is achieved through self-assembly of alkane phosphates or alkane phosphonates from aqueous solutions (assembly system A) in combination with prepatterned surfaces whereas only one type of the prepattern area (a) forms a molecularly assembled layer A of alkane phosphates, while the other prepattern area(s) (β, . . . ) remains uncoated (“selective chemical reactivity contrast”).

Another preferred example is a device of where α is an oxide, nitride or carbide of a metal that chemically interacts with phosphates and/or phosphonates, in particular transition metal oxides such as titanium oxide, tantalum oxide, niobium oxide, zirconium oxide, or non-transition metal oxides that chemically interact with phosphates or phosphonates, and where b is an oxide that does not interact, in particular silicon oxide.

Another preferred example is s device of where the specificity is achieved through assembly of polyionic, PEG-grafted polymers (B) from aqueous solution at a pH chosen such that one of the two or more prepattern areas (e.g. β) is charged oppositely in comparison to the polyionic copolymer and becomes coated by the copolymer due to electrostatic interactions, while the other prepattern area(s) (e.g. α) at the same pH carries a charge of same sign as the copolymer and does not or does less become coated (“electrostatic contrast”).

Another preferred example is a device where the prepattern area β is an oxide, nitride or carbide with an isoelectric point (IEP) that is lower than that of area α and the assembly system is a (at the pH of application) polycationic copolymer and the pH of the assembly system solution is chosen between the IEP of area α and area β.

Another preferred example is a device where the prepattern area β is an oxide, nitride or carbide with an isoelectric point (IEP) that is higher than that of area α and the assembly system is a (at the pH of application) polyanionic copolymer and the pH of the assembly system solution is chosen between the IEP of area α and area β.

Another preferred example is a device where the specificity is achieved through self-assembly of a di- or multiblock copolymer with hydrophobic and hydrophilic segments interacting with a substrate where one of the prepattern area (α) is more hydrophobic than the remaining areas, and therefore gets coated by the di- or multiblock copolymer A (“hydrophobic-hydrophilic contrast”) while the other prepattern area (β) remains uncoated or less coated (schematic process scheme in FIG. 9).

Another preferred example is a device where the di- or multiblock copolymer is a polypropylene oxide (PPO)-poly(ethylene glycol) (PEG) copolymer imparting protein resistance to the more hydrophobic surface.

Another preferred example is a device where the hydrophobic prepattern area (α) is composed of a hydrophobic polymer or of an oxide that has been hydrophobized through silanization or application of an alkane phosphate self-assembly system, while the hydrophilic prepattern area is either composed of a hydrophilic polymer or is an inherently hydrophilic oxide or is an oxide that has been made permanently hydrophilic through application of a self-assembled monolayer using a molecule with hydrophilic terminal functional group.

Another preferred example is a device where in a second molecular assembly step B the prepattern area β that has not been coated with the alkane phosphate becomes coated with a protein-resistant polymeric layer, leading to a final pattern that is interactive with a biological environment (proteins, cells) in areas A and not interactive (“protein- and cell-resistant”) in areas B.

Another preferred example is a device where B is the assembly of a polyionic PEG coated copolymer, adsorbing onto the oppositely charged area β, e.g. polycationic poly(L-lysine)-g-poly(ethylene oxide) adsorbing at pH of between 2 and 8 onto negatively charged silicon oxide.

Another preferred example is a device where in a second step the prepattern area α becomes coated with a functionalized polyionic PEG-grafted copolymer A through application of the second self-assembly solution at a pH different from step 1, at which pH the area α is now oppositely charged in comparison to the polyionic copolymer A and becomes coated with the functionalized polymer, leading to a final pattern that is interactive with a biological environment (proteins, cells) in areas A and non-interactive (“protein- and cell-resistant”) in areas B.

Another preferred example is a device where the polyionic PEG-grated copolymer is functionalized at the end of the PEG chains through covalent linkage to a biologically active group such as biotin interacting with streptavidin, or a peptide or a protein, interacting specifically with receptors in cell membranes.

Another preferred example is a device where in a second step the more hydrophilic area (β) that has not been coated in assembly step A gets coated in the second assembly step B with a molecule that induces specific or non-specific interaction with the biological environment.

Another preferred example is a device where B is a functionalized polyionic PEG-grafted copolymer that interacts electrostatically with the oppositely charged surface β or is an alkane phosphate that turns the area β into a hydrophobic, non-specifically interactive area resulting in a final interactive/non-interactive pattern.

Another preferred example is a device where a oligo(ethylene oxide) functionalized alkane phosphate is used as the molecular assembly system A, leading to a non-interactive area A, while the area β are subsequently treated with an assembly system B that renders this area interactive, e.g. by adsorbing a functionalized, polyionic PEG-grafted co-polymer.

Another preferred example is a device where a functionalized (e.g. biotin or peptide or reactive chemical group attached at end of PEG chains) PPO-PEG diblock or PEG-PPO-PEG triblock, or multiblock copolymer is used to render the correspondingly covered area specifically interactive, followed by a second assembly system that renders the remaining area non-interactive, e.g. through adsorption of a polyionic PEG-coated copolymer.

Another preferred example is a device where after application of assembly system A and B the resulting interactive/non-interactive pattern is further modified through selective treatment of area A and/or B with biochemically or biologically relevant molecules.

Another preferred example is a device where the selective treatment is a nonspecific adsorption of proteins or other biomolecules to the area that is (non-specifically) interactive, e.g. hydrophobic or a selective interaction with ligands previously immobilized in step A or B, e.g. streptavidin interacting specifically with biotin ligand on one of the pattern area.

Another preferred example is a device where living cells are added to patterned surfaces and become immobilized selectively on one of the pattern area, through interaction with selectively and nonspecifically adsorbed protein or proteins, or through specific interactions with bioligands such as peptides or proteins that have in a previous step been immobilized through covalent attachment to one of the pattern areas.

Another, preferred subject of the invention is a bioanalytical sensing platform comprising a “device with chemical surface pattern” according to any of the embodiments disclosed above and at least one biological or biochemical or synthetic recognition element, for the specific recognition and/or binding of one or more analytes and/or for the specific interaction with said analyte(s), immobilized either directly or mediated by a self-assembled layer and/or by an adhesion-promoting layer on at least one of the different types of regions a or b or . . . .

It is preferred that the biological or biochemical or synthetic recognition element is attached to at least one of the applied self-assembly systems A or B, or adsorbs on at least one of said self-assembly systems.

It is further preferred that the biological or biochemical or synthetic recognition elements are immobilized in a one-or two-dimensional array of discrete measurement areas, wherein a single discrete measurement area is defined by the area occupied by said immobilized biological or biochemical or synthetic recognition elements on an individual, closed region a or b.

Up to 1,000,000 measurement areas can be provided in a two-dimensional arrangement on one “device with chemical surface pattern”, and a single measurement area can occupy an area between 10⁻⁴ mm² and 10 mm².

It is preferred that the measurement areas are arranged at a density of at least 10, preferably of at least 100, most preferably of at least 1000 measurement areas per square centimeter.

The biological or biochemical or synthetic recognition elements can be selected from the group comprising proteins, such as mono- or polyclonal antibodies or antibody fragments, peptides, enzymes, aptamers, synthetic peptide structures, glycopeptides, oligosaccharides, lectins, antigens for antibodies (e.g. biotin for streptavidin), proteins functionalized with additional binding sites (“tag proteins”, such as “histidin-tag proteins”), nucleic acids (such as DNA, RNA, oligonuelotides or polynucleotides) and nucleic acid analogues (such as peptide nucleic acids, PNA) or their derivatives with artificial bases, soluble, membrane-bound proteins, such as membrane-bound receptors and their ligands. Also whole cells or cell fragments can be immobilized for specific recognition and detection of one or more analytes.

It is preferred, that whole cells or cell fragments, for determination of different analytes, are immobilized in discrete measurement areas.

It is desired to minimize the amount of biological material required for the detection of a certain analyte. The amount of necessary material is dependent on the sensitivity of the detection step. It is desired that less than 100, preferably less than 10, most preferably only 1-3 cells or cell fragments be immobilized per measurement area.

Many embodiments of a bioanalytical sensing platform according to the invention are characterized in that said sensing platform is operapable for analyte determination by means of a label, which is selected from the group comprising luminescence labels, especially luminescent intercalators or “molecular beacons”, absorption labels, mass labels, especially metal colloids or plastic beads, spin labels, such as ESR and NMR labels, and radioactive labels.

On the other side, refractive methods do not necessarily require the use of a label. In this context, methods for generation of surface plasmon resonance in a thin metal layer on a dielectric layer of lower refractive index can be included in the group of refractive methods, if the resonance angle of the launched excitation light for generation of the surface plasmon resonance is taken as the quantity to be measured. Surface plasmon resonance can also be used for the amplification of a luminescence or the improvement of the signal-to-background ratios in a luminescence measurement. The conditions for generation of a surface plasmon resonance and the combination with luminescence measurements, as well as with waveguiding structures, are described in the literature, for example in U.S. Pat. No. 5,478,755, No. 5,841,143, No. 5,006,716, and No. 4,649,280.

In this application, the term “luminescence” means the spontaneous emission of photons in the range from ultraviolet to infrared, after optical or other than optical excitation, such as electrical or chemical or biochemical or thermal excitation. For example, chemiluminescence, bioluminescence, electroluminescence, and especially fluorescence and phosphorescence are included under the term “luminescence”.

In case of the refractive measurement methods, the change of the effective refractive index resulting from molecular adsorption to or desorption from the waveguide is used for analyte detection. This change of the effective refractive index is determined, in case of grating coupler sensors, for example from changes of the coupling angle for the in- or outcoupling of light into or out of the grating coupler sensor, in case of interferometric sensors from changes of the phase difference between measurement light guided in a sensing branch and a referencing branch of the interferometer. In case of a device for the generation of surface plasmon resonance, the change of the effective refractive index can be determined from a change of the resonance angle at which a surface plasmon (in a thin metal film deposited on a dielectric substrate) is generated. If a tunable excitation light source, both for a grating coupler sensor and for a device for generation of a surface plasmon resonance, a change of the effective refractive index can also be determined from a change of the excitation wavelength for satisfying the respective resonance condition, when the excitation light is launched at a fixed angle close to the resonance angle.

Therefore, certain embodiments of a bioanalytical sensing platform according to the invention are characterized in that they are operapable for analyte determination by means of the detection of a change of the effective refractive index in the near field of the surface of said sensing platform due to molecular adsorption on or desorption from said sensing platform.

Specific embodiments of a bioanalytical sensing platform according to the invention are operapable for analyte determination by means of the detection of a change of the conditions for generation of a surface plasmon in a metal layer being part of said sensing platform, wherein said metal layer preferably comprises gold or silver. It is preferred that said metal layer has a thickness between 40 nm and 200 nm, still more preferably between 40 nm and 100 nm.

The aforesaid refractive methods have the advantage, that they can be applied without using additional marker molecules, so-called molecular labels. The disadvantage of these label-free methods, however, is, that the achievable detection limits are limited to pico- to nanomolar concentration ranges, dependent on the molecular weight of the analyte, due to lower selectivity of the measurement principle, which is not sufficient for many applications of modern trace analysis, for example for diagnostic applications.

Lower detection limits can be achieved, for example using methods based on luminescence detection, especially if these methods are combined with optical waveguide techniques, for example by fluorescence excitation in the evanescent field of an optical waveguide.

Therefore, preferred embodiments of a bioanalytical sensing platform according to the invention are characterized in that said sensing platform is operapable for analyte determination by means of the detection of a change of one or more luminescences.

A bioanalytical sensing platform according to such an embodiment can be operapable to receive excitation light in an epi-illumination configuration.

It is preferred that the material of a bioanalytical sensing platform according to the invention, which material is in contact with the measurement areas, is transparent, at least at one excitation wavelength, to a depth of at least 200 nm, measured from the surface supporting the immobilized biochemical or biological or synthetic recognition elements in said measurement areas.

Characteristic for another embodiment of a bioanalytical sensing platform according to the invention is, that it is operapable to receive excitation light in an transmission-illumination configuration.

In general, it is preferred that the materials of said sensing platform are transparent at least one excitation wavelength.

Characteristic for a preferred embodiment of a bioanalytical sensing platform according to the invention is, that it is operapable as an optical waveguide. It is further preferred that said optical waveguide is essentially planar.

For such an embodiment of a bioanalytical sensing platform operapable as an optical waveguide, it is preferred that it comprises an optically transparent (i.e. optically transparent at least one excitation wavelength) material selected from the group comprising silicates, such as glass or quartz, thermoplastic or moldable plastics, such as polycarbonates, polyimides, acrylates, especially polymethyl methacrylates, and polystyrenes.

It is especially preferred that a bioanalytical sensing platform according to the invention comprises an optical thin-film waveguide with a layer (a) being optically transparent at least one excitation wavelength on a layer (b) being optically transparent at least at the same excitation wavelength, wherein the refractive index of layer (b) is lower than the one of layer (a).

In order to couple excitation light into the wave guiding layer of a bioanalytical sensing platform based on an optical waveguide, said waveguiding layer is in optical contact to at least one of the optical coupling elements selected from the group comprising prism couplers, evanescent couplers formed by joined optical waveguides with overlapping evanescent fields, distal end (front face) couplers with focusing lenses, preferably cylindrical lenses, located in front of a distal end (front face) of the waveguiding layer, and coupling gratings.

It is preferred that light incoupling into the optically transparent layer (a) is performed by means of one or more grating structures (c) formed in layer (a).

It is further preferred that outcoupling of light guided in the optically transparent layer (a) is performed by means of one or more grating structures (c′) formed in layer (a), and wherein grating structures (c′) can have the same or different grating period as optional additional grating structures (c).

Characteristic for one type of bioanalytical sensing platforms according to the invention, with coupling gratings (c) for incoupling of excitation light into the waveguiding layer (a) is, that an array of at least 4 regions with at least two different “prefabricated patterns” a and b and, optionally, with one or more self-assembly systems (A, B, . . . ) deposited on the different “prefabricated patterns”, is located after an incoupling grating (c), with respect to the direction of propagation of light guided in layer (a) after its incoupling by said grating.

Characteristic for another type of bioanalytical sensing platforms according to the invention, with coupling gratings (c) and/or (c′) is, that an array of at least 4 regions with at least two different “prefabricated patterns” a and b and, optionally, with one or more self-assembly systems (A, B, . . . ) deposited on the different “prefabricated patterns”, is located on a coupling grating (c) or (c′).

For some applications it is preferred that a continuous coupling grating (c) or (c′) extends over at least 30% of the surface of said sensing platform.

The optically transparent layer (b) should be characterized by low absorption and fluorescence, in the ideal case free of absorption and fluorescence. Additionally, the surface roughness should be low, because the surface roughness of the layer (b) does affect, dependent on the deposition process to a more or less large extent, the surface roughness of an additional layer (a) of higher refractive index, when it is deposited on layer (a) as a waveguiding layer. An increased surface roughness at the boundary (interface) layers of layer (a) leads to increased scattering losses of the guided light, which, however, is undesired. These requirements are fulfilled by numerous materials.

It is preferred that the material of the second optically transparent layer (b) comprises an optically transparent material (i.e. optically transparent at least at one excitation wavelength) selected fro the group comprising silicates, such as glass or quartz, thermoplastic or moldable plastics, such as polycarbonate, polyimides, acrylates, especially polymethylmethacrylates, and polystyrenes.

For a given layer thickness of the optically transparent layer (a), the sensitivity of an arrangement according to the invention increases along with an increase of the difference between the refractive index of layer (a) and the refractive indices of the adjacent media, i.e., along with an increase of the refractive index of layer (a). It is preferred, that the refractive index of the first optically transparent layer (a) is higher than 1.8.

Another important requirement on the properties of layer (a) is, that the propagation losses of the light guided in layer (a) should be as low as possible. It is preferred, that the first optically transparent layer (a) comprises a material selected from the group comprising TiO₂, ZnO, Nb₂O₅, Ta₂O₅, HfO₂, or ZrO₂, preferably especially from the group comprising TiO₂, Ta₂O₅, and Nb₂O₅. Combinations of several such materials can also be used.

For a given material of layer (a) and given refractive index, the sensitivity does increase with decreasing layer thickness, up to a certain lower limiting value of the layer thickness. The lower limiting value is determined by the cut-off of light guiding, if the layer thickness falls below a threshold value determined by the wavelength of the light to be guided, and by an observable increase of the propagation losses in very thin layers, with further decrease of their thickness. It is preferred, that the thickness of the first optically transparent layer (a) is between 40 and 300 nm, preferably between 70 and 200 nm.

If an autofluorescence of layer (b) cannot be excluded, especially if it comprises a plastic such as polycarbonate, or for reducing the affect of the surface roughness of layer (b) on the light guiding in layer (a), it can be advantageous, if an intermediate layer is deposited between layers (a) and (b). Therefore, it is characteristic for another embodiment of the bioanalytical sensing platform according to the invention, that an additional optically transparent layer (b′) with lower refractive index than and in contact with layer (a), and with a thickness of 5 nm-10 000 nm, preferably of 10 nm-1000 nm, is located between the optically transparent layers (a) and (b).

It is preferred, that the grating structures (c) and optional additional grating structures (c′) have a period of 200 nm-1000 nm and a grating modulation depth of 3 nm-100 nm, preferably of 10 nm-30 nm.

Thereby it is preferred, that the ratio of the modulation depth to the thickness of the first optically transparent layer (a) is equal or smaller than 0.2.

The grating structures can be provided in different forms (geometry). It is preferred, that a grating structure (c) is a relief grating with any profile, such as right-angular, triangular or semi-circular profile, or a phase or volume grating with a periodic modulation of the refractive index in the essentially planar optically transparent layer (a).

Further embodiments of sensing platforms, which can be incorporated into a bioanalytical sensing platform according to the invention if they are provided with a “chemical surface pattern” as described above, as well as methods for analyte determination performed with these sensing platforms, are disclosed in U.S. Pat. Nos. 5,822,472, 5,959,292, and U.S. Pat. No. 6,078,705, and in the patent applications WO 96/35940, WO 97/37211, WO 98/08077, WO 99/58963, PCT/EP 00/04869, and PCT/EP 00/07529. Therefore, the embodiments disclosed therein are also part of this invention and incorporated by reference.

Another subject of the invention is a method for the simultaneous qualitative and/or quantitative determination of one or more analytes in one or more samples, wherein said samples are brought into contact with the measurement areas on a bioanalytical sensing platform according to the invention, and wherein the resulting changes of signals from said measurement areas are measured.

For many applications methods are preferred wherein said changes of signals from the measurement areas are obtained upon using a label, which is selected from the group comprising luminescence labels, especially luminescent intercalators or “molecular beacons”, absorption labels, mass labels, especially metal colloids or plastic beads, spin labels, such as ESR and NMR labels, and radioactive labels.

Other embodiments of a method for analyte determination according to the invention are characterized in that analyte determination is performed upon detection of a change of the effective refractive index in the near field of the surface of said sensing platform due to molecular adsorption on or desorption from said sensing platform.

A special method is based on the detection of a change of the conditions for generation of a surface plasmon in a metal layer being part of said sensing platform, wherein said metal layer preferably comprises gold or silver.

For most applications, however, methods are preferred, wherein analyte determination is performed upon detection of a change of one or more luminescences.

The excitation light from one or more light sources can be launched on the bioanalytical sensing platform in a configuration of epi-illumination. In another embodiment of said method, excitation light from one or more light sources is launched on the bioanalytical sensing platform in a configuration of transmission-illumination.

Preferred are embodiments of a method for analyte determination according to the invention, wherein the bioanalytical sensing platform comprises an optical waveguide, which is preferably essentially planar, and wherein excitation light from one or more light sources is coupled into said waveguide by means of an optical coupling element selected from the group comprising prism couplers, evanescent couplers formed by joined optical waveguides with overlapping evanescent fields, distal end (front face) couplers with focusing lenses, preferably cylindrical lenses, located in front of a distal end (front face) of the waveguiding layer, and coupling gratings.

Especially advantageous is an embodiment, wherein said bioanalytical sensing platform comprises an optical thin-film waveguide, with a first optically transparent layer (a) on a second optically transparent layer (b) with lower refractive index than layer (a), wherein furthermore excitation light is incoupled into the optically transparent layer (a) by one or more grating structures formed in the optically transparent layer (a), and directed, as a guided wave, to the measurement areas located thereon, and wherein furthermore the luminescence from molecules capable to luminesce, which is generated in the evanescent field of said guided wave, is detected by one or more detectors, and wherein the concentration of one or more analytes is determined from the intensity of these luminescence signals.

In the method disclosed above, (1) the isotropically emitted luminescence or (2) luminescence that is incoupled into the optically transparent layer (a) and outcoupled by a grating structure (c) or luminescence comprising both parts (1) and (2) can be measured simultaneously.

For the generation of the luminescence or fluorescence in the method according to the invention, a luminescence or fluorescence label can be used, that can be excited and emits at a wavelength between 300 nm and 1100 nm.

The luminescence or fluorescence labels can be conventional luminescence or fluorescence dyes or also so-called luminescent or fluorescent nano-particles based on semiconductors [W. C. W. Chan and S. Nie, “Quantum dot bioconjugates for ultrasensitive nonisotopic detection”, Science 281 (1998) 2016-2018].

The luminescence label can be bound to the analyte or, in a competitive assay, to an analyte analogue or, in a multi-step assay, to one of the binding partners of the immobilized biological or biochemical or synthetic recognition elements or to the biological or biochemical or synthetic recognition elements.

Additionally, a second or more luminescence labels of similar or different excitation wavelength as the first luminescence label and similar or different emission wavelength can be used. Thereby, it is advantageous, if the second or more luminescence labels can be excited at the same wavelength as the first luminescence label, but emit at other wavelengths.

For other applications, it is advantageous, if the excitation and emission spectra of the applied luminescent dyes do not overlap or overlap only partially.

In the method according to the invention, it can also be advantageous, if charge or optical energy transfer from a first luminescent dye acting as a donor to a second luminescent dye acting as an acceptor is used for the detection of the analyte.

In addition, it can be of advantage, if besides determination of one or more luminescences, changes of the effective refractive index on the measurement areas are determined. It can be of further advantage, if the one or more luminescences and/or determinations of light signals at the excitation wavelengths are performed polarization-selective. The method allows also for measuring the one or more luminescences at a polarization that is different from the one of the excitation light.

The method according to the invention, according to any of the embodiments disclosed above, allows for the simultaneous or sequential, quantitative or qualitative determination of one or more analytes of the group comprising antibodies or antigens, receptors or ligands, chelators or “histidin-tag components”, oligonucleotides, DNA or RNA strands, DNA or RNA analogues, enzymes, enzyme cofactors or inhibitors, lectins and carbohydrates.

The samples to be examined can be naturally occurring body fluids, such as blood, serum, plasma, lymphe or urine, or egg yolk.

A sample to be examined can also be an optically turbid liquid or surface water or soil or plant extract or bio- or process broths.

The samples to be examined can also be taken from biological tissue.

A further subject of the invention is the use of a bioanalytical sensing platform and/or of a method for analyte determination, both according to any of the embodiments disclosed above, for quantitative or qualitative analysis for the determination of chemical, biochemical or biological analytes in screening methods in pharmaceutical research, combinatorial chemistry, clinical and preclinical development, for real-time binding studies and the determination of kinetic parameters in affinity screening and in research, for qualitative and quantitative analyte determinations, especially for DNA- and RNA analytics, for the generation of toxicity studies and the determination of expression profiles and for the determination of antibodies, antigens, pathogens or bacteria in pharmaceutical product development and research, human and veterinary diagnostics, agrochemical product development and research, for patient stratification in pharmaceutical product development and for the therapeutic drug selection, for the determination of pathogens, nocuous agents and germs, especially of salmonella, prions and bacteria, in food and environmental analytics.

In the area of biosensors, in particular in case of bioaffinity sensors relying on the specific detection of biologically relevant molecules or molecular assemblies (e.g., DNA, RNA, proteins, cell receptors, etc.), patterning techniques are already playing a crucial role in the design of microarray sensor chips, which allow for more than one type of analyte to be analyzed on the same chip through controlled spatial arrangement of recognition units In particular, in microarray sensor chips with a total number of specific sensing areas (equivalent to “measurement areas” or “spots”) in the order of 10² to 10⁵ measurement areas per cm², the perfect spatial arrangement and localization of active recognition units (i.e. biological or biochemical or synthetic recognition elements) in the measurement areas is mandatory for high quality (low probability of faulty measurements, high degree of quantitativeness of the analytical measurement), reproducible performance. The stringent control over the spatial arrangement of active units becomes more important as the feature size and spacing decreases. Such microarray sensors are generally fabricated by spatially controlled immobilization of the biological or biochemical or synthetic recognition elements on a chemically homogeneous chip surface using techniques such as ink jet printing, microdroplet capillary spotting, and others. Furthermore, there is a growing interest in cell-based sensors, i.e. sensors where living cells are attached in a controlled fashion to chip surfaces and are used to sense environmental factors or, more specifically, cellular responses to their exposure to chemicals.

Critical issues for the bioaffinity and cell-based microarray sensor chips are
The precise location of the spots and their arrangement in a regular geometric pattern
The uniformity of the spot size over the whole array
The preservation of the activity of the biological or biochemical or synthetic recognition elements
The homogeneity of the distribution of the recognition units within the spot area e.g. avoidance of “doughnut structures”), and
The low signal (e.g. fluorescence) background of the area immediately adjacent to the array spot.

The invented SMAP processes is able to provide solutions to a number of frequently observed specific problems and challenges that are encountered in practice and that are related to the above-mentioned critical issues, for example:
The SMAP process is able to produce patterns with geometrically organized areas of different wetability, e.g. areas of any geometric form, size and interarray spacings (pitch) that are hydrophilic in a “sea” or “background” that is hydrophobic. This has two positive consequences: Firstly, independently of the spotting technique used, the (aqueous) droplet, once it has landed at the chip surface, is precisely located on the wettable area since it “jumps into contact” due to the hydrophobic surrounding area. Therefore, the precision of spot localization can be higher than that of the spotting technique itself (which is influenced by the mechanics of the system, drop formation, size and detachment, electrostatic effects, etc.) and is basically determined by the precision of the SMAP pattern. The latter is extremely accurate. Secondly, the spot geometry after drying of the spotted droplet can be precisely controlled, since the contact area between droplet and chip surface is controlled mostly through the size of the hydrophilic area; it can be easily optimized for a given droplet volume.
Through the precise control of the droplet-chip interfacial area, the surface-to-volume ratio of the landed droplet can be precisely controlled. This is an important aspect, since it allows a certain control over the evaporation rate (which increases with increasing surface/volume ratio). Evaporation rate is important if the process of immobilization of the spotted recognition units takes some time, for example in case where a chemical bond between the recognition unit and functional chemical groups at the chip surface has to be formed within the time of liquid-surface contact. Secondly, if both the droplet volume (through the spotting technique) and the droplet-surface contact area (through application of the invented SMAP technique can be controlled, one can achieve within the spotted area α much better control over the homogeneity of the spatial distribution of the recognition units adsorbed at the surface after evaporation of the droplet. If this ratio is not well controlled, one often experiences inhomogeneous distribution of the recognition unit, e.g. a higher concentration in the center of the spot, or a “donut shape” with higher concentrations at the border of a (e.g. round spot). Such deviations from a perfectly homogeneous, controlled size spot adversely affects both the adequate quantification of the spot signal (e.g. fluorescence) and the maximum attainable detection sensitivity.

For cell-based sensors, the precise placement of cells on geometrically well-controlled, cell-adhesive spots is highly relevant [Chen C S, Mrksich M, Huang S, et al., Geometric control of cell life and death, Science 276 (5317): 1425-1428, May 30, 1997]. The SMAP technique allows the production of such well-controlled cell-adhesive patterns while at the same time ensuring a very low tendency for cells to attach outside the adhesive areas (i.e., in the non-adhesive areas). Since the functionality of the cell is influenced by its morphology, a precise control over the cell-surface contact area is a factor that is essential for the performance of the cell-based chip. Furthermore, the type and density of attachment sites for cells (e.g. peptides interacting with cell membrane receptors, focal contacts) are essential for both the attachment strength and cellular activity such as differentiation of the cell) [Rezania A, Healy K E, The effect of peptide surface density on mineralization of a matrix deposited by osteogenic cells, J Biomed Mater Res (4): 595-600, Dec. 15, 2000]. The SMAP technique is an ideal technique for producing cell-adhesive patterns on cell-based sensor chips of high geometric fidelity, control over the surface density of biological functions interacting with the cell, and interfacial stability over time.

In terms of the chip technology and transducer requirements, the SMAP technique has the advantage of flexibility with respect to the choice of the appropriate substrate materials. The patterns can be produced on preferably transparent substrates or chips, e.g. by using transparent metal-oxide-based coatings on transparent substrates (glass, quartz, etc.). This allows one to use optical transmission technique for the control of the patterns and for the use of optical detection techniques such as optical transmission (fluorescence) microscopy or optical evanescent field technique (e.g. optical waveguide techniques). Alternatively, the SMAP technique can be applied to non-transparent, e.g. metallic reflecting chip surfaces. This may be advantageous if detection techniques requiring reflective surfaces such as reflection microscopy or evanescent field techniques requiring metal surface coatings (e.g. surface plasmon resonance methods) are to be used.

There are a number of arguments why chemically patterned surfaces of tailored interactiveness with the environment are of interest to applications in the area of biomaterials, biomedical devices and implants.

Surfaces that are patterned into geometrically ordered areas that are adhesive to proteins and/or cells with a non-adhesive background will interact with a biological medium in a more controlled and predictable way than is the case with homogeneous (unpatterned) or randomly heterogeneous surfaces.

One argument for the exploitation of patterns in the size range of cells (few to few tens of micrometers) on biomaterials and implants is the fact that the size of cells (projection of cell shape on surface) can be influenced by the size (area) of the adhesive pattern. The size of the cell-surface contact area on the other hand has been shown to affect the development of the cells [Chen C S, Mrksich M, Huang S, et al., Geometric control of cell life and death, Science 276 (5317): 1425-1428, May 30, 1997]. For example, proliferation and differentiation activity of osteoblastic cells react differently (mostly oppositely) to the dimensions of the cell-adhesive area and there is a particular size of the cell-surface contact area for which differentiation of osteoblasts cells is fastest [Thomas C H, et al., J. Biomech. Eng. 121: 40, 1999; Thomas C H, et al. Proceedings of the Society for Biomaterial Conference, Hawaii, 2000, p. 1222]. Furthermore, the form of the cells and the formation of stress fibres can be tailored by choosing appropriate patterns of dimensions which contain both features with dimensions similar to those of cells (e.g. 5 to 100 μm) as well as connected or disconnected features with features in the low micrometer (e.g. 1-5 μm) or submicrometer range, representative of subcellular features such as membrane receptors or focal contacts. Since the stress fibres are important for cellular activity, not only the static behavior but also the dynamics of cells, e.g. motility, can be steered by appropriate patterns. In particular, anisotropic patterns may be used to either direct cell motility along certain directions on the surface of an implant or to impose anisotropy on the properties of the cellular or tissue interface that forms with time at the implant surface. Such anisotropic properties at the interface may be beneficial to the short and/or long-term performance of the biomaterial body or biomaterial-cell culture interface, for example in case of bone-related implants or in tissue engineering of boneous material in vitro or in vivo (natural bone is a highly anisotropic material).

In a given situation in the body or in a primary cell culture, different cells coexist and interact with the surface of the artificial material. It may therefore be of interest to the bioengineer to develop patterns that have a positive influence on the behavior of different types of cells at the surface. For example, in case of a bone implant, it may be advantageous to have patterns that strongly support the attachment and differentiation of osteoblasts, but not of fibroblasts, in order to favor the formation of a boneous, rather than fibrous, interfacial tissue. This may be achieved by choosing an optimum size and form of the adhesive pattern, an optimum distance between the features within the pattern and an optimum symmetry of the arrangement of the adhesive areas within features. Another form would be to choose an interactive biological functionality within the adhesive pattern that interacts more strongly with one type of cells that with other types. As an example, it has been demonstrated that heparin-binding peptides of the type . . . KRSR . . . interact more strongly (almost selectively) with osteoblasts than with fibroblast, while the integrin-binding peptide of type . . . RGD . . . interacts strongly with both types of cells [Hasenbein M E, Anderson T T, Bizios R, Proceedings of the Society for Biomaterials Conference, Hawaii, 2000, p.110; Dee K C, et al., Tissue Engineering 1 (1995) 135; Dee K C, et al., J. Biomed. Mater. Res. 25 (1991) 771]. One could similarly envisage patterns that interact with cells that are important for healing, tissue integration and stability of implants, while such patterns do not support the attachment and proliferation of bacteria.

Another application for chemically patterned surfaces is related to the cell type that is relevant in almost all in vivo implant applications, the macrophage. While macrophages fulfill an important function in “cleaning up” implantation sites and implant surfaces during the healing phase, their extended actions, in particular the occurrence of frustrated phagocytosis and formation from macrophages of multinuclear giant cells (“foreign body giant cells”, FBGC), may lead to sustained inflammation and retarded or prevented healing reactions. Chemically patterned surfaces could improve the situation in at least two different ways: a) if the surface of an implant is patterned into cell-adhesive and non-adhesive areas in dimensions significantly smaller than the size of an attached macrophage, the latter is expected to be prevented from developing a tight seal between the cell membrane and the surface. As a consequence, the macrophage (and osteoclast)—typical excluded volume cannot form, which is a prerequisite for the sustained action of generated, destructive acids, superoxides and peroxides within this excluded electrolyte volume. Therefore, an unfavorably massive degree of chemical attack of biomaterials through macrophage activity could be prevented by using cell-adhesive/non-adhesive patterns of suitable geometry. The same mechanism would hold for the action of osteoclasts in a bone environment. In a different approach (that can be combined with the first one), pattern geometries can be designed that restrict macrophage cells to individual sites at the surface, well separated from each other. In such a situation, unfavorable FBGC formation would be suppressed or at least reduced compared to a homogeneous or randomly heterogeneous surface.

In summary, patterns may allow the biomedical engineer, interested in designing implants or tissue engineering constructs with improved performance, to influence not only the type and density of cells at the biomaterial-body or biomaterial tissue interface, but also on the development of cells at the interface with time, and therefore also on the kinetics of formation and the properties of the resulting interfacial tissue, which forms adjacent to the patterned biomaterial or implant surface. While the chemical pattern is basically two-dimensional, its effect in the biomaterial and tissue engineering area can be three-dimensional, exerting its influence also in the third dimension, i.e. perpendicular to the surface, and up to distances much larger than the pattern dimensions.

### BRIEF DESCRIPTION OF THE DRAWINGS

The invention is further described below by providing information on the general procedures of the selective molecular assembly patterning technique and detailed by way of specific examples and drawings as follows:

FIG. 1. Regular, geometric (a) and random, statistically distributed (b) patterns with chemical composition A in a background of composition B. Surface view (a) and (b); cross section (c) without (top) and with (bottom) topographical change between A and B.

FIG. 2. Scheme of the technological steps in the fabrication of chemical patterns using the SMAP process. See text for discussion.

FIG. 3. Scheme for the production of patterned substrates by lithography to be used in the context of the SMAP technique.

FIG. 4. Patterning of substrates in the submicron dimension range using small particles such as nanosized polystyrene colloidal particles.

FIG. 5. Preparation of substrates for the SMAP process based on chemical contrast “written” with the help of a focused ion beam.

FIG. 6. Surface chemical composition of the prepatterned substrate surfaces (in cross section) used for the examples describing the application of the SMAP technique below.

FIG. 7. Fluorescence microscopy image of the SMAP treated surface showing in dark gray the SiO₂ areas (5×5 μm) that are protein-resistant due to the selective adsorption of PLL-g-PEG, while the light gray areas are selectively covered by the fluorescently labeled protein streptavidin that adsorbs to the TiO₂ areas which previously had been hydrophobized by an alkane phosphate (DDP) self-assembled monolayer.

FIG. 8. Si-Wafer, coated with 90 nm TiO₂, and 12 nm SiO₂. Prepatterned surface produced by dry etching of structures with dimensions 10×15 □m (central rectangle) and 2×15 □m lines. SMAP steps: 1) DDP, 2) PLL-g-PEG-biotin, 3) Albumin fluorescently labeled with Oregon Green, 4) Streptavidin fluorescently labeled with Texas Red (details are described in the text). There is selective adsorption of the fluorescently labeled albumin to the hydrophobic DDP areas, while fluorescently labeled streptavidin binds to the biotinylated PLL-g-PEG, but not to the albumin-passivated DDP areas.

FIG. 9. Adsorption of PLL-g-PEG-biotin to TiO₂ and SiO₂ surfaces respectively, as a function of pH of the molecular assembly solution. The adsorbed mass of PLL-g-PEG-biotin was judged by quantitative fluorescence microscopy upon exposure of the PLL-g-PEG-biotin-treated surfaces to Oregon-Green-labeled streptavidin. The difference in pH dependence of the molecular adsorption process between the two surfaces forms the basis for the SMA patterning technique with contrast resulting from electrostatic interaction (“electrostatic contrast”, type II).

FIG. 10. Schematic drawing of SMAP according to type III, using hydrophobic-hydrophilic contrast.

FIG. 11. List of selected combinations of prepatterning techniques with molecular assembly processes. Standard micro- and nano-patterning methodologies are listed on the left. Their objective is to produce a specimen with two kinds of surfaces present, thus providing a material contrast for the SMAP patterning. Various examples of the latter are listed on the right. DDP: dodecylphosphate or -phosphonate. Hb: hydrophobic anchoring group. X, Y—specific receptors (examples include biotin, RGD-peptide, etc.).

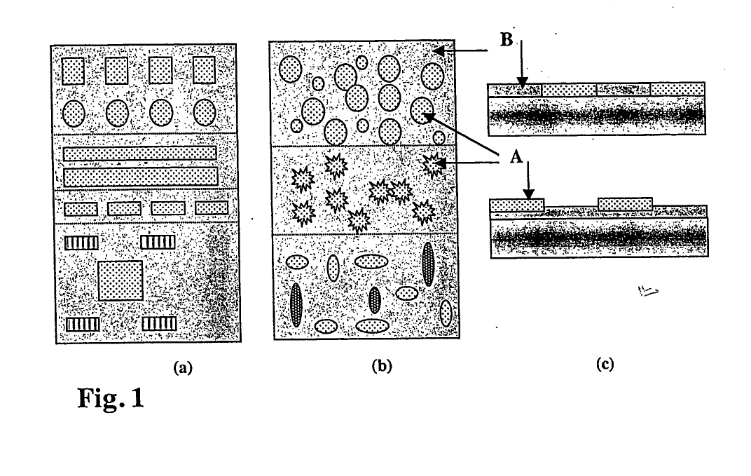

### DETAILED DESCRIPTION

The technological basis for the Selective Molecular Assembly Patterning (SMAP) is based on selective, spontaneous assembly out of solution of molecules with a physico-chemical, biochemical or biological functionality onto a substrate surface that contains a suitable pattern prefabricated using any of the state-of-the-art surface-structuring techniques. The chemical structure of the prefabricated substrate pattern is chosen such that the subsequent molecular assembly step selectively modifies one type of pattern, generally followed by a second assembly process to coat the second type of pattern. Further selective modification steps may follow until the desired patterned surface or interface architecture has been achieved.

### General Flow Diagram for Creating Patterned Surfaces Based on SMAP

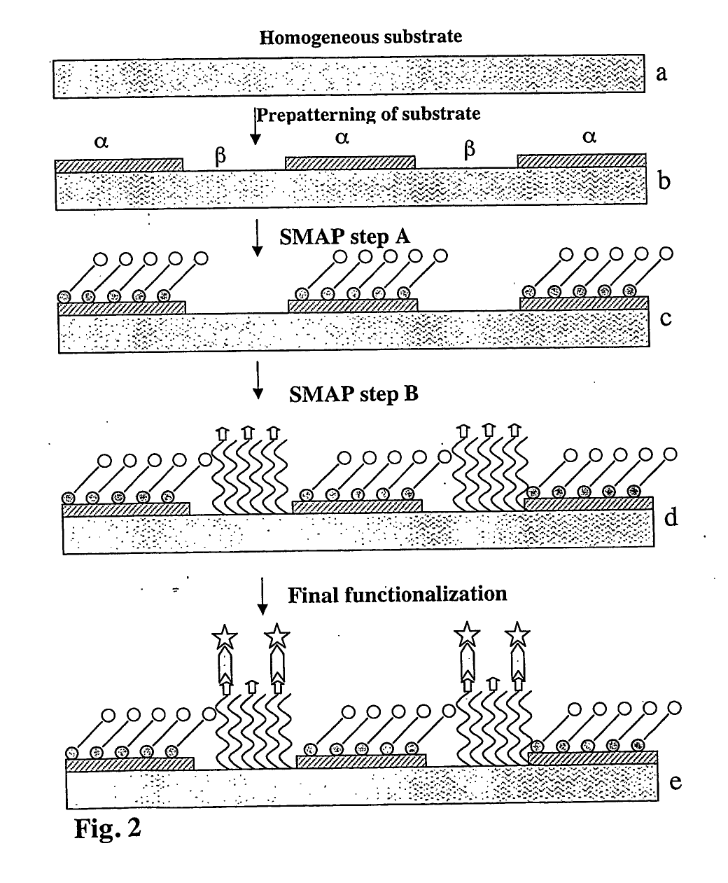

FIG. 2 schematically represents a typical sequence for the application of the SMAP technique (surface architecture shown in cross section):
(a) Substrate with homogeneous properties as the starting material.
(b) Prepatterning of substrate into areas α and β with different chemical composition (=generation of material contrast) using state-of-the-art structuring/patterning techniques. It should be noted, that this chemical patterning must not be necessarily be associated with a topographical patterning, as shown in FIG. 2, but can also be performed within the plane of the surface, e.g. using local chemical modification upon exposure to laser light.
(c) Application of a spontaneous molecular assembly system that forms an adlayer selectively on area β (SMAP step A), but does not (or to a much lower degree) interact with area α.
   (d) Application of a spontaneous molecular assembly system that forms an adlayer selectively on area α (SMAP step B).
   (e) Depending on the system, one or several further functionalization steps may be applied to complete the desired surface or interface architecture.

Depending on the type of molecules and their degree of functional properties chosen in SMAP step A and B, the surface at stage (d) may already contain the final functionality needed for the given application. Alternatively, the surface at stage (d) may contain functional groups in areas A or B that can be converted (preserving the spatial selectivity) into the desired functionality in one or several additional modification steps (e).

### Examples of Molecular Assembly Systems, Suitable for the SMAP Technique

Three types of molecular assembly processes are described as specific examples that are suitable for use in the Selective Molecular Assembly Patterning technique. They exploit a specific response to a particular set of physicochemical properties of the prepatterned substrate:
Type I: specific covalent or complex-coordinative binding, i.e. “contrast based on selective chemical reactivity”.
Type II: attractive versus repulsive electrostatic interactions, i.e. “electrostatic contrast”.
Type III: van der Waals interactions of hydrophobic molecular segments with hydrophobic areas at the surface, i.e. exploiting “hydrophobic-hydrophilic contrast”.
   Type I: SMAP Using Alkane Phosphate Self-Assembled Monolayers (“Selective Chemical Reactivity Contrast”):

Alkane phosphates and alkane phosphonates have been described in the literature [M. Textor, L. Ruiz, R. Hofer, A. Rossi, K. Feldman, G. Hähner, N. D. Spencer, Structural Chemistry of Self-Assembled Monolayers of Octadecylphosphoric Acid on Tantalum Oxide Surfaces, Langmuir 16 (7): 3257-3271 (2000)] to self-assemble on oxide surfaces such as tantalum oxide, titanium oxide, niobium oxide or aluminum oxide forming partially ordered monolayers with well-defined physico-chemical properties. Their application as homogenous (unpatterned) surfaces to the biomaterial and biosensor field has been described in Swiss priority patent application No. CH 1732/00. If aqueous solutions of alkane phosphates are used, it has been observed that SAMs are formed on a variety of metal oxides such as tantalum oxide, titanium oxide and niobium oxide, but NOT on silicon oxide. The silicon oxide surface remains uncoated. Therefore, if a prestructured substrate surface is used that contains, for example, a pattern with silicon oxide patches and with titanium (or niobium or tantalum) oxide patches, only the titanium (or tantalum or niobium) oxide areas get coated with the alkane phosphate. If a methyl-terminated alkane phosphate such as dodecyl phosphate (DDP) is used, a high contrast in wetability results with hydrophobic areas corresponding to TiO₂-DDP, while the uncoated silicon oxide patches remain hydrophilic. In a second step, the silicon oxide patches may be coated with a different molecular assembly system, e.g. by adsorption of protein-resistant poly(L-lysine)-g-poly(ethylene glycol) (PLL-g-PEG) on the SiO₂ pattern, wherein g denotes the ratio between the number of lysine units and the number of poly(ethylene glycol) side chains, or by alkane phosphate with a different terminal functional group on the SiO₂ pattern by adsorption from the corresponding organic solvents solution.

A corresponding example is given in Example 1 below.

Type II: SMAP Using Polyionic Polymers (“Electrostatic Contrast”)

Polyionic copolymers have been shown to assemble spontaneously on charged surfaces forming stable adlayers due to electrostatic (and other types of) interactions if the charge of the polymer and of the surface are opposite (as described in patent application WO 00/65352). For example poly-L-lysine, which is positively charged at neutral pH, adsorbs to negatively charged surfaces—such as tissue-culture polystyrene, or metal oxide surfaces such titanium oxide or silicon oxide. The use of polyethylene glycol grafted, polyionic copolymers is particularly useful for the biosensor and biomaterial area, since they form stable monolayers resistant to protein adsorption. This is important if the objective is to eliminate non-specific interactions in general and impose specific interactions to certain areas of the pattern.

One typical way of exploiting the electrostatic contrast is to choose a prepatterned oxide surfaces based on two different oxides with substantially different isolelectric points (IEPs). By adjusting the pH of the molecular assembly solution such that the two types of pattern area are oppositely charged, conditions can be found for which the polyionic polymer coats only one type of metal oxide, i.e. the one that is oppositely charged in comparison to the polyionic polymer. In a second step, the area that has not been coated in the first assembly step serves as a substrate for the self-assembly of a different polymer, or of the same polymer with an additional functional group.

A corresponding example is given in Example 2 below.

Type III: SMAP Using Hydrophobic-Hydrophobic Interactions (“Hydrophobic-Hydrophilic Contrast”)

Functional copolymers that contain at least one segment that is highly hydrophobic can be used within the SMAP technology if the prepatterned surface contains hydrophobic and hydrophilic areas. Such copolymers will strongly interact with the hydrophobic areas due to the hydrophobic effect and via van der Waals (“hydrophobic-hydrophobic”) interactions. Although such polymers may also cover hydrophilic areas through weaker physical interactions, their binding strength is likely to be weak enough to be removed by a solvent and suitable rinsing conditions, effectively resulting in a pattern that contains the polymer only in the hydrophobic areas. Copolymers of the “Pluronic” containing hydrophobic segments composed of poly(propylene oxide) and hydrophilic segments composed of poly(ethylene glycol) form a typical class of molecules that are suitable for the SMAP technique in combination with prepatterned hydrophobic/hydrophilic surfaces. The PEG chains in the Pluronics molecules can be further functionalized with e.g. a biochemical or biological functionality.

A corresponding example of the application of PEG-PPO-PEG within the SMAP technology is given in Example 3 below.

### Prepatterned Substrate Fabrication

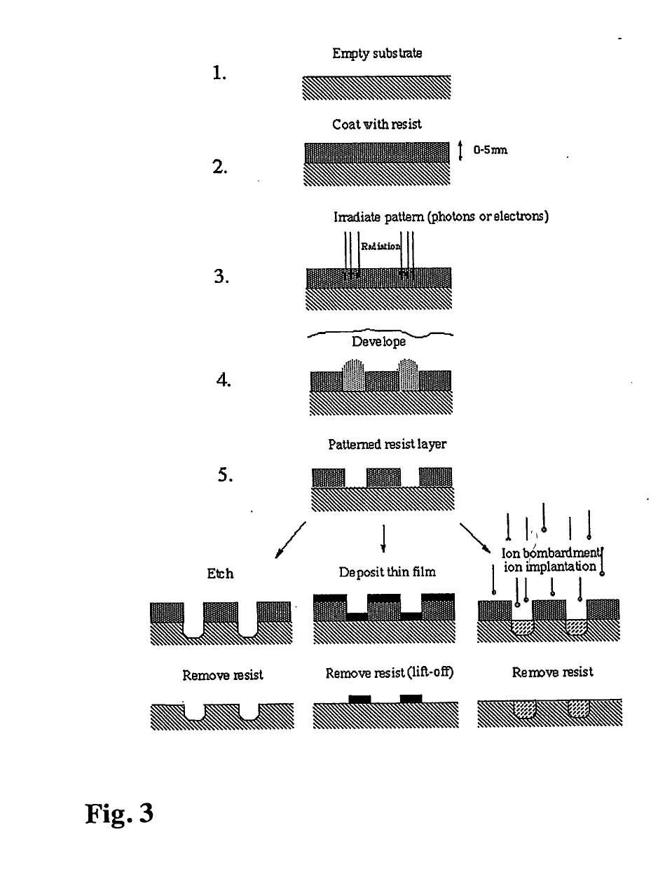

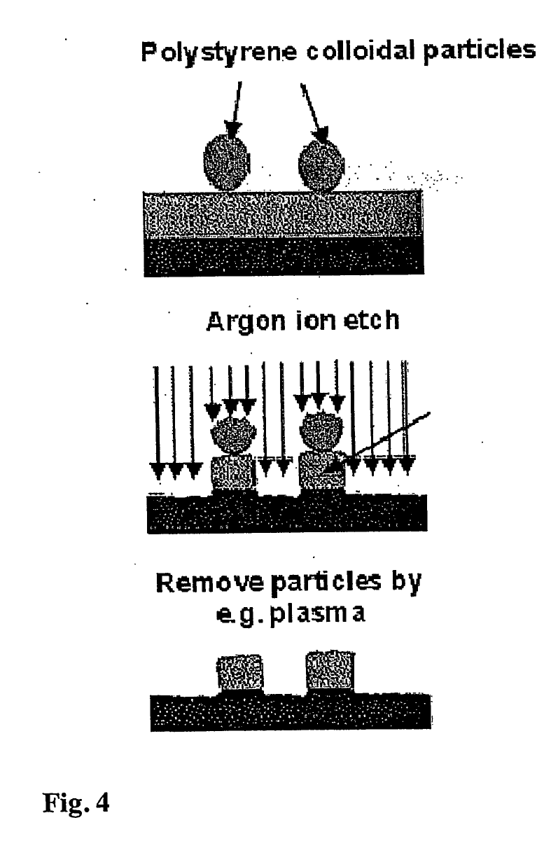

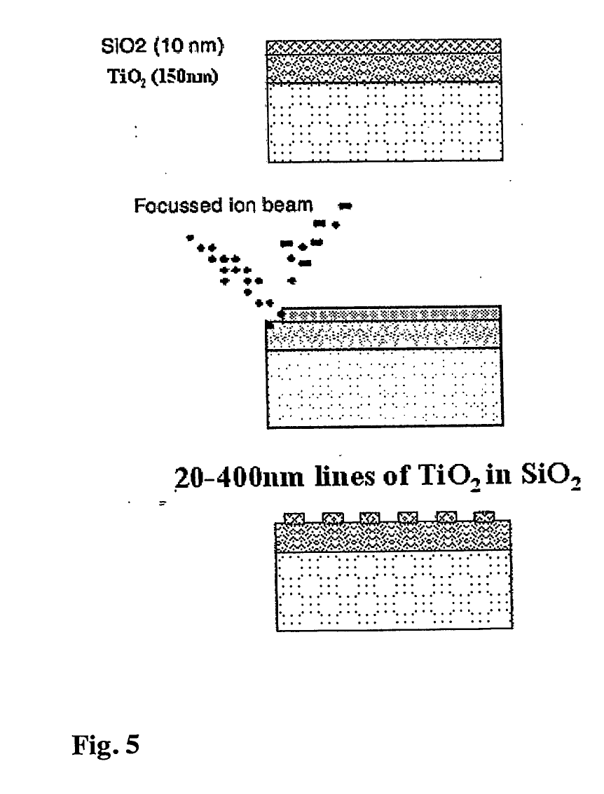

A variety of state-of-the-art techniques are principally suitable to prepattern substrates that are subsequently used in combination with the novel SMAP technique. In particular, the following techniques can be used:
Photolithography using masks and photoresist coatings on suitable substrates: standard lithography using visible, UV or X-ray exposure, or more recently developed techniques such as interference-based lithographic structuring [Rogers, J. A., Paul, K. E., Jackman, R. J., Whitesides, G. M. Generating ˜90 nm features using near-field contact-mode photolithography with an elastomeric phase mask. J. Vac. Sci. Technol. B16(1), (1998) 59-68.s]. A typical procedure using conventional lithography is shown in FIG. 3.
Electron-beam lithography using masks and photoresist coatings on suitable substrates (similar to FIG. 3, but with sequential writing of the surface structures using an electron beam).
Lithographic techniques using colloids deposited onto surfaces, schematically shown in FIG. 4 [Rogers, J. A., Paul, K. E., Jackman, R. J., Whitesides, G. M. Generating ˜90 nm features using near-field contact-mode photolithography with an elastomeric phase mask. J. Vac. Sci. Technol. B16(1), (1998) 59-68.s].
Focused ion beam in combination with a thin-film-deposited substrate according the FIG. 5.

These techniques differ in terms of the range of feature sizes that can be produced, parallel versus sequential “writing” of the patterns, costs, applicability to non-flat (e.g. curved) surfaces and requirements for the selection of suitable substrates. Depending on the envisaged surface structure and application, a preferred technique from the list above or any technique that allows one to chemically pattern surfaces can be chosen and applied to fabricate the prepatterned substrate to be used in the subsequent SMAP process.

### Specific Examples of the SMAP Technique

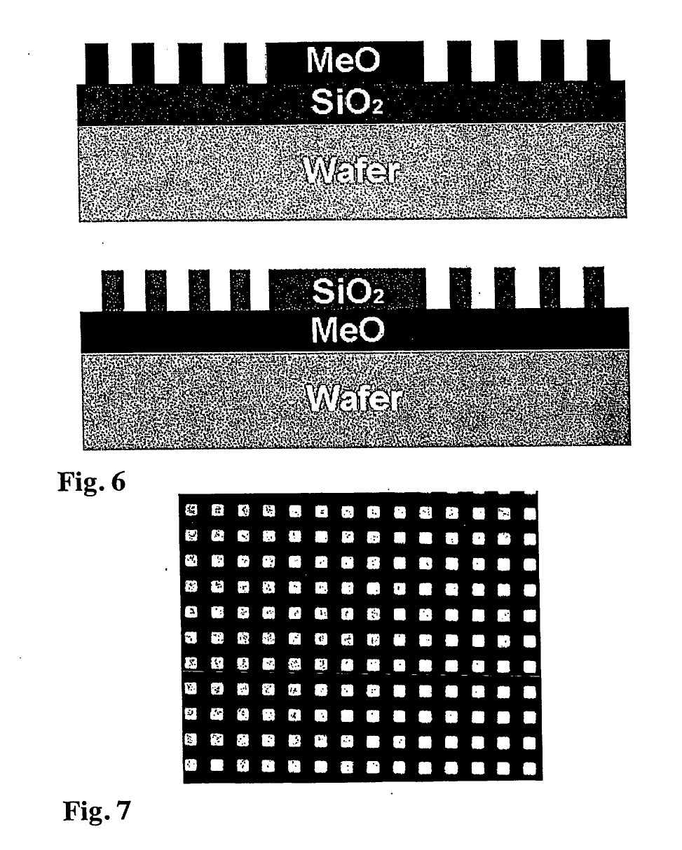

Three specific examples are presented in the following. In terms of the first molecular assembly step (SMAP step A in FIG. 2), they are based on SMAP process of type I, II and III respectively. The substrate for these examples of SMAP-patterning has one of the structure shown in FIG. 6, where MeO stands for the appropriate transition metal oxide, such as titanium oxide, niobium oxide, tantalum oxide, or aluminum oxide, etc., while SiO₂ stands for silicon oxide.

### EXAMPLE 1

### SMAP Based on Alkane Phosphate//Poly(L-lysine)-g-poly(ethylene oxide) System (“Selective Chemical Reactivity Contrast”)

Out of aqueous solutions, dodecyl phosphate (DDP) self-assembles on metal oxides but not on silicon oxide. Subsequent application of PLL-g-PEG renders silicon oxide protein resistant, hence creating a pattern of protein-adhesive and resistant areas. Protein adsorption to the DDP-modified metal oxide surface is in this case non-specific and due to hydrophobic interactions between the hydrophobic alkane phosphate SAM and hydrophobic moieties of the protein.

As a specific example, the following consecutive steps were applied:
a) The starting surface is produced using photolithography according to general scheme in FIG. 3. A silicon wafer was first coated with 100 nm TiO₂ followed by 10 nm SiO₂ using the magnetron sputtering technique. After application of a photoresist coating, irradiation through a corresponding mask, dry etching through the SiO₂ layer using CF₄/CF₃H gas mixture and removal of the photoresist, a pattern of 5×5 μm squares of TiO₂ was produced while the rest of the surface remains SiO₂ (as shown in FIG. 6 bottom).
b) The lithographically patterned TiO₂/SiO₂ surface is then dipped in an aqueous solution of the ammonium salt of dodecyl phosphoric acid (DDP, 0.5 mole/L) for 24 h at room temperature (RT). A self-assembled monolayer of DDP forms on top of the TiO₂ 5×5 □m areas, rendering these areas highly hydrophobic.
c) The surface is carefully rinsed using high purity water
d) The surface is exposed by dipping for 15 min into an aqueous solution (in HEPES buffer) of poly(L-lysine)-g-poly(ethylene glycol) (PLL-g-PEG; MW of PLL: 20,000 Da, g=3.5, MW of PEG: 2,000 Da; concentration of PLL-g-PEG=1 mg/mL; for details see: [G. L. Kenausis, J. Vörös, D. L. Elbert, N. P. Huang, R. Hofer, L. Ruiz, M. Textor, J. A. Hubbell, N. D. Spencer, Poly(L-lysine)-g-poly(ethylene glycol) Layers on Metal Oxide Surfaces: Attachment Mechanism and Effects of Polymer Architecture on Resistance to Protein Adsorption, J. Phys. Chem. B 104: 3298-3309 (2000)].
e) The surface is carefully rinsed using high purity water.
f) The surface is exposed by dipping to fluorescently (Texas Red)-labeled streptavidin in HEPES buffer for 60 min at RT.
g) Final washing in HEPES buffer and in high purity H₂O.

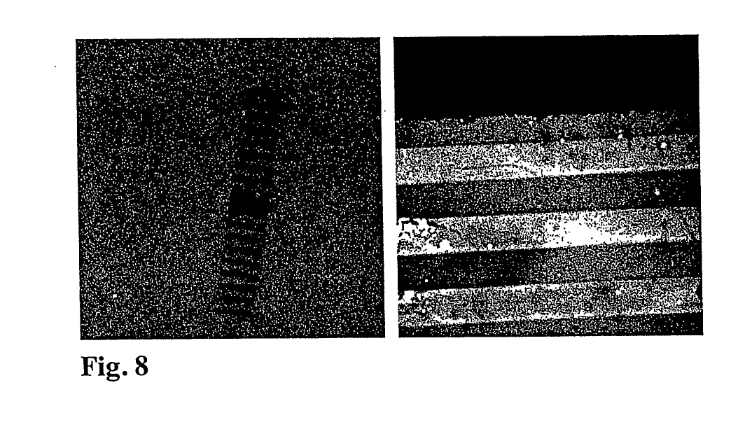

FIG. 7 shows a fluorescence microscopy image of the SMAP treated surface showing in dark gray to black the SiO₂ areas that are protein-resistant due to the selective adsorption of PLL-g-PEG, while the light gray to white areas are selectively covered by the protein streptavidin (fluorescently labeled) that adsorbs to the TiO₂ areas which previously had been hydrophobized by an alkane phosphate self-assembled monolayer.

FIG. 8 demonstrates that the same SMAP process also works with more complex patterns.

This pattern has been produced in the following way: A silicon wafer was coated by physical vapor deposition with 90 nm TiO₂, followed by 12 nm SiO₂. A photoresist was applied and a pattern with a central square of 10×15 □m and lines of dimension 2×15 □m etched into the SiO2 layer. The SMAP process consisted of the following sequential steps:
a) A hydrophobic dodecyl phosphate self-assembled monolayer (DDP) was formed from aqueous solution (concentration: 0.5 mM, RT) of the ammonium salt of DDP (dipping time: 24 h).
b) After rinsing, a PLL-g-PEG/PEG-biotin was deposited by dipping the sample into an aqueous solution of PLL-g-PEG/PEG-biotin (PLL-g-PEG; MW of PLL: 20,000 Da, g=3.5, MW of PEG: 2,000 Da, MW of PEG-biotin: 3,200 Da; 50% of PEG chains functionalized with biotin; concentration of PLL-g-PEG/PEG-biotin=1 mg/mL).
c) The sample was exposed to an aqueous solution of Oregon-Green-labeled albumin (concentration: 20 □g/mL; exposure time: 1 h). Albumin covers selectively the hydrophobic areas, i.e. the TiO₂-DDP pattern, but does not cover the protein-resistant PLL-g-PEG/PEG-biotin areas.
d) Finally, the sample was exposed to a solution of Texas-Red-labeled streptavidin (concentration: 1 mg/mL; exposure time: 1 h) resulting in specific interactions with the biotin functional groups of the PLL-g-PEG/PEG-biotin in the SiO₂ pattern areas, while the albumin passivated areas are resistant to (further) protein adsorption.
e) The resulting strong chemical contrast shown in the fluorescence microscopy image of FIG. 8 is due to albumin above the TiO₂ areas and streptavidin above the SiO₂ areas.

### EXAMPLE 2

### SMAP Based on Poly(L-lysine)-g-poly(ethylene glycol)//Poly(L-lysine)-g-poly(ethylene oxide-biotin) System (“Electrostatic Contrast”)

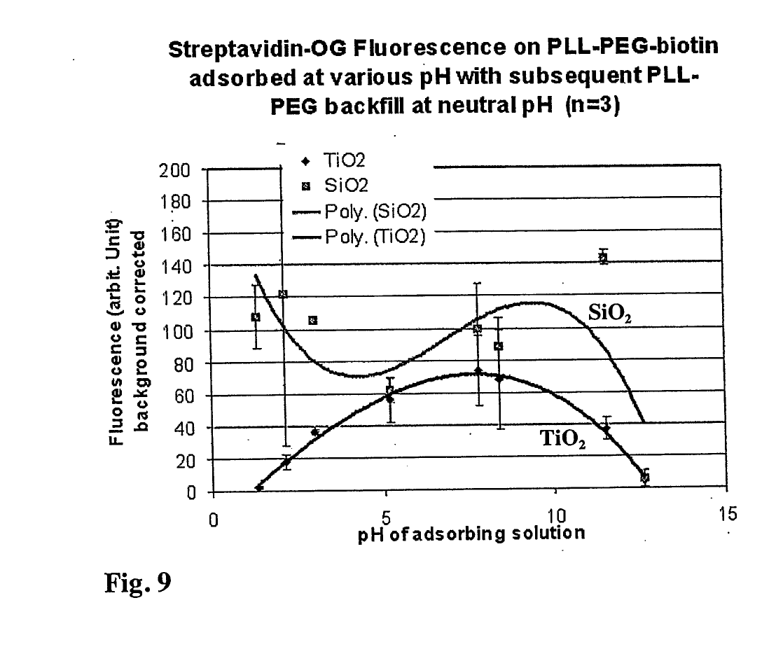

The difference in the isoelectric point between titanium oxide and silicon oxide can be exploited to produce SMAP type II patterns based on electrostatic contrast. This type of SMAP is illustrated using the spontaneous assembly of poly(L-lysine)-g-poly(ethylene glycol) at charged surfaces, which is governed by electrostatic interactions. This technique requires a starting surface with a pattern formed by two materials whose isoelectric points (IEP) are sufficiently different (IEP of SiO₂: ca. 2.5; IEP of TiO₂: ca. 6). FIG. 9 shows the dependence of the adsorbed mass of PLL-g-PEG(-biotin) (MW of PLL: 20,000 Da, g=3.5, MW of PEG: 2,000 Da) to SiO₂ and TiO₂ surfaces respectively, as a function of the pH of the PLL-g-PEG aqueous solution to which the surfaces were exposed for a time of 15 min. It is obvious from FIG. 9 that at pH=1.2, PLL-g-PEG (or functionalized PLL-g-PEG) can be selectively adsorbed to the SiO₂ region, while the TiO₂ surface at the same pH remains uncovered due to the repulsive interactions between the positively charged TiO2 surface and the positively charged PLL-g-PEG.

The following protocol is a typical example of exploiting the differences in IEP and creating a pattern of areas that allow the area-selective immobilization of streptavidin through streptavidin-biotin interactions. It involves the use the of a patterned TiO₂/SiO₂ substrate, followed by area-selective, spontaneous adsorption of biotin-functionalized PLL-g-PEG (PLL-g-PEG/PEG-biotin) to the SiO₂ pattern from aqueous solution at pH=1.2, followed by backfilling the bare oxide areas (particularly the TiO2 pattern) at a pH of 7 with (non-functionalized) PLL-g-PEG. The produced pattern can then be further modified by area-selective immobilization of streptavidin to the PLL-g-PEG/PEG-biotin areas. Such a surface can for example be used as a substrate for the immobilization of biotinylated antibodies to the streptavidin sites for application in protein sensing (proteomics) or for the defined localization and attachment for cells in the area of cell-based sensing.

Detailed SMAP Protocol:
Patterned SiO₂/TiO₂ surface is exposed to an aqueous solution of PLL-g-PEG/PEG-biotin (concentration=1 mg/mL) at a pH of 1.2 and at RT. Due to the surface charges at this pH, it only adsorbs to the SiO₂ areas, while the TiO₂ areas, being strongly positively charged, remain uncoated after this step.
Exposure of the surface to an aqueous solution of (unmodified) PLL-g-PEG (concentration=1 mg/mL) at RT and at pH=7, “backfilling” the TiO₂ areas and potentially present defects in the PLL-g-PEG/PEG-biotin coating on SiO₂.

### EXAMPLE 3

### SMAP Based on PEG-PPO-PEG//Poly(L-lysine)-g-poly(ethylene oxide-biotin) System (“hydrophobic-hydrophilic contrast”)

Example 3 relies on another type of contrast, namely the exploitation of the hydrophobic/hydrophilic contrast already described in Example 1. In a further step, the hydrophobic areas are made protein- and cell-resistant (“non-interactive”) via the interaction with a triblock molecule that contains both a hydrophobic block (to interact with the hydrophobic area of the pattern) and hydrophilic PEG blocks to render the adlayer protein-resistant. The other area of the pattern, the PLL-g-PEG/PEG-X, is cell-interactive due to X=specific peptides interacting with cell membrane receptors.

Protocol:
(a) MeO-DDP patches can be modified with PEG-based copolymers possessing a hydrophobic backbone (PPO-PEG). This renders metal oxide-DDP areas protein resistant. PLL-g-PEG/PEG-X (where X stands for a specific receptor functionality, such as biotin or RGD peptide) is used in the subsequent step to render silicon oxide able to bind desired macromolecules, specifically and with high affinity.
(b) The pattern of non-adhesive and specific areas can be inverted by using a hydrophobic backbone-PEG copolymer bearing a functional group.
(c) A combination of hydrophobic backbone-PEG bearing one functional group and PLL-g-PEG bearing another can be used, creating a pattern of doubly-adhesive areas. The adhesion is in this case specific in nature (as opposed to (a).

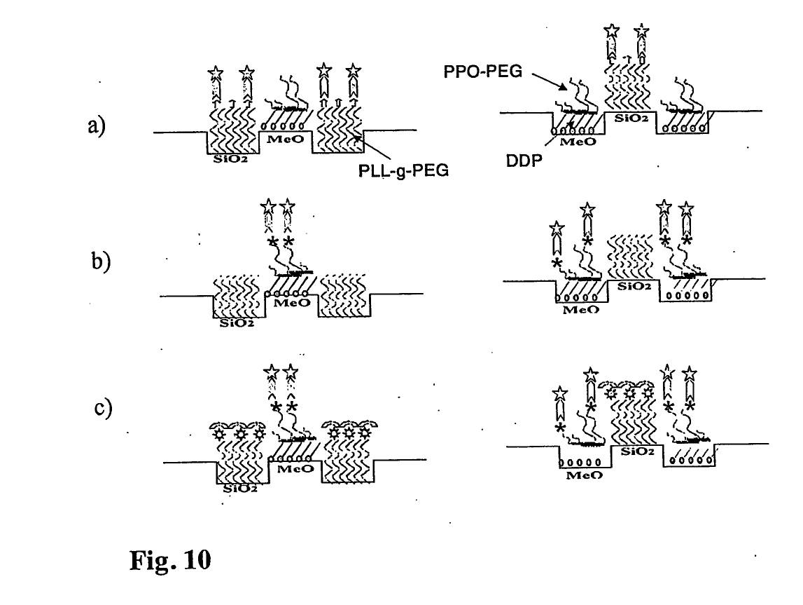

FIG. 10 illustrates schematically the SMAP according to type III, using hydrophobic-hydrophilic contrast.

### Overview of Further Examples Combining Different Prepatterning and SMAP-Based Techniques

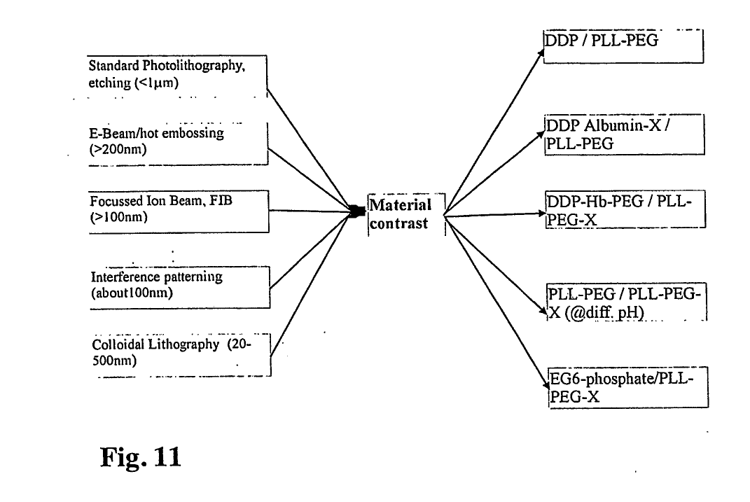

FIG. 11 summarizes a selected, not exhaustive number of possibilities of combining prepatterning techniques to produce metal or oxide patterns together with molecular assembly patterning to produce biologically-relevant chemical contrast using the SMAP technique.

Standard micro- and nano-patterning methodologies and recently developed techniques such as colloidal lithography and interference patterning are listed on the left. Their objective is to produce a specimen with two kinds of surfaces present, thus providing a material contrast for the SMAP patterning according to one of the three SMAP contrast methods discussed above. Apart from the specific oxide patterns TiO₂/SiO₂, many other oxide combinations are suitable for the SMAP process. Their selection depends on the requirement of the SMAP process (i.e. coordinative interactions with alkane phosphates, surface charge for interaction with polyionic copolymers, etc.) and of the application (e.g. requirement for optical transparency, etc.). Apart from SiO₂ and Al₂O₃, transition metal oxides often have the necessary properties to be used as at least one type of the prepattern material for later SMAP application. Also, metal surfaces, e.g. bulk metal specimens or metal films deposited onto suitable substrates can also be used as materials for prepatterning, since most of the metals are covered by an oxide film, MeₓO_{y}, which can serve as one component for the prepatterning step.

In terms of the molecular assembly processes suitable to be combined within the SMAP process, various examples are listed on the right of FIG. 11. These represent a selection of preferred assembly techniques, but many other techniques are compatible with the SMAP process as long as they fulfill the requirements for one of the three types of SMAP processes.

Additionally, Table 1 compiles a more extensive list of molecular assembly techniques suitable for applications within the SMAP process. The table lists the type/class of molecules, their interaction with specific examples of non-metal oxides, metal oxides or metals (with a natural or artificially produced oxide film at the surface) in terms of the binding or immobilization type, the non-interactiveness of the surface following the assembly step (resistance to biomolecule adsorption and cell attachment) or the non-specific interactiveness towards biomolecules (e.g. proteins) and cells or the (bio)specific interactiveness (either directly after the corresponding assembly process or after an additional functionalization step).

Table 1 is also not exhaustive. Many more types of prepatterned substrate materials including metallic surfaces, nonmetallic, inorganic surfaces (oxides, carbides, nitrides, etc.) or polymeric materials can be used as long as they fulfill one or several of the requirements for applications in the SMAP technology of type I, II or III (see above).

Similarly many more molecular assembly techniques than just the selected examples discussed above can be used in combination with suitable prepatterned surfaces, as long as they interact in a predictable way with a particular type of prepatterned surface and react selectively with one type of the prepatterned areas and renders such a surface either non-interactive, interactive in a non-specific way, or interactive in a (bio)specific way.

TABLE 1
Compilation of molecular assembly systems
suitable for applications within the SMAP process (examples).
The table lists the type/class of molecules, their interaction
with specific examples of materials in terms of the binding or
immobilization type, the non-interactiveness of the surface
following the assembly step (resistance to biomolecule adsorption
and cell attachment) or the non-specific interactiveness
towards biomolecules (e.g. proteins) and cells or the
(bio)specific interactiveness (either directly after the corresponding
assembly process or after an additional functionalization
step).

| | | | Further |
|---|---|---|---|
| | Interaction | Subsequent interaction | biochemical |
| Molecule | with substrate | with | or biological |
| (type, example) | (type, examples) | biological medium | functionalization |
| Alkane phosphates | Adsorbs | a) Non-specific | — |
| or | onto: transition | adsorption of | b) Modification |
| phosphonates | metal oxides | protein(s) | through |
| (CH₃— | such | through hydrophobic- | specific |
| terminated), | as oxides | hydrophobic interactions. | interaction |
| e.g. CH₃—(CH₂)ₓ—PO₄ | of Ti, Nb, | b) Protein- and | with functionalized |
| with | Zr, Ta; | cell-resistant | albumin |
| x = 2-24 | other metal | surface after | surface, |
| | oxides that | passivation with | e.g. between |
| | form metal- | albumin or functionalized | streptavidin |
| | phosphate | albumin, | and |
| | complexes | e.g. biotinylated | biotinylated |
| | such as | albumin | albumin |
| | aluminum | c) Protein- and | c) Modification |
| | oxide. | cell-resistant | through |
| | Does NOT | surface after | specific |
| | adsorb onto | adsorption of | interaction |
| | silicon oxide | di- or multi- | with functionalized |
| | | block polymer | PEG-PPO. |
| | | with hydrophobic | |
| | | segments and hydrophilic, | |
| | | non- | |
| | | interactive segments, | |
| | | e.g. PEG- | |
| | | PPO (‘Pluronics’, | |
| | | see Example | |
| | | 3). The PEG | |
| | | chains can be | |
| | | further functionalized, | |
| | | e.g. | |
| | | with biotin. | |
| Oligo- or | Adsorbs | Resistant to | — |
| poly(ethylene | onto: transition | protein adsorption | |
| oxide)- | metal oxides | and cell | |
| modified alkane | such | attachment | |
| phosphates | as oxides | | |
| or | of Ti, Nb, | | |
| phosphonates, | Zr, Ta; | | |
| e.g. (EO)_{y}—(CH₂)ₓ—PO₃ | other metal | | |
| with | oxides that | | |
| x = 2-24, y = 2-50 | form metal- | | |
| | phosphate | | |
| | complexes | | |
| | such as | | |
| | aluminum | | |
| | oxide. | | |
| | Does NOT | | |
| | adsorb onto | | |
| | silicon oxide | | |
| Oligo- or | Adsorbs | Resistant to | Biospecific |
| poly(ethylene | onto: transition | protein adsorption | attachment |
| oxide)- | metal oxides | and cell | of antigen, |
| modified alkane | such | attachment | e.g. streptavidin |
| phosphates | as oxides | | to |
| or | of Ti, Nb, | | biotin or |
| phosphonates, | Zr, Ta; | | of cells |
| with terminal | other metal | | through |
| (□-positioned) | oxides that | | specific |
| biological | form metal- | | peptide- |
| ligand, e.g. | phosphate | | cell membrane |
| biotin or peptide | complexes | | interactions. |
| | such as | | |
| | aluminum | | |
| | oxide. | | |
| | Does NOT | | |
| | adsorb onto | | |
| | silicon oxide | | |
| Oligo- or | Adsorbs | Resistant to | Biological |
| poly(ethylene | onto: transition | protein adsorption | moiety can |
| oxide)- | metal oxides | and cell | be attached |
| modified alkane | such | attachment | to functional |
| phosphates | as oxides | | group |
| or | of Ti, Nb, | | through covalent |
| phosphonates, | Zr, Ta; | | bond, e.g., |
| with terminal | other metal | | peptide, |
| (□-positioned) | oxides that | | protein, |
| reactive | form metal- | | enzyme. |
| chemical | phosphate | | |
| group, e.g. N- | complexes | | |
| hydroxysuccinimidyl, | such as | | |
| maleimide, vinylsulfone, | aluminum | | |
| | oxide. | | |
| | Does NOT | | |
| | adsorb onto | | |
| | silicon oxide | | |
| Polycationic. | Adsorbs to | Protein- and | — |
| PEG-modified | oxide surfaces | cell-resistant | |
| copolymers, | with | surface | |
| e.g. PLL-g-PEG | negative | | |
| (see Examples | surface | | |
| 1 and 2) | charge, | | |
| | i.e. at a | | |
| | solution pH | | |
| | that is | | |
| | higher than | | |
| | the | | |
| | isoelectric | | |
| | point of | | |
| | the oxide | | |
| | surface, | | |
| | e.g. at pH > 5 | | |
| | for | | |
| | TiO₂ or at | | |
| | pH > 1.5 | | |
| | for SiO₂. | | |
| | Does NOT | | |
| | (or less) | | |
| | adsorb to | | |
| | positively | | |
| | charged | | |
| | surfaces. | | |
| Polycationic. | Adsorbs to | Protein- | Interacts |
| PEG-modified | oxide surfaces | resistant. | specifically |
| copolymers, | with | | with |
| e.g. PLL-g-PEG | negative | | cells (if |
| (see Examples | surface | | ligand = specific |
| 1 and 2), with | charge, | | peptide), |
| part or all of | i.e. at a | | or with DNA |
| the PEG chains | solution pH | | or RNA if |
| functionalized | that is | | ligand is |
| with a bioactive | higher than | | an oligonucleotide |
| ligand | the | | or |
| | isoelectric | | with streptavidin |
| | point of | | if |
| | the oxide | | ligand = biotin. |
| | surface, | | |
| | e.g. at pH > 5 | | |
| | for | | |
| | TiO₂ or at | | |
| | pH > 1.5 | | |
| | for SiO₂. | | |
| | Does NOT | | |
| | (or less) | | |
| | adsorb to | | |
| | positively | | |
| | charged | | |
| | surfaces. | | |
| Polyanionic | Adsorbs to | Protein- and | — |
| PEG-modified | oxide surfaces | cell-resistant | |
| copolymers, | with | | |
| e.g. | positive | | |
| Poly (glycolic | surface | | |
| acid)-g-PEG | charge, | | |
| (see Examples | i.e. at a | | |
| 1 and 2) | solution pH | | |
| | that is | | |
| | lower than | | |
| | the | | |
| | isoelectric | | |
| | point of | | |
| | the oxide | | |
| | surface, | | |
| | e.g. at pH > 5 | | |
| | for | | |
| | TiO₂ or at | | |
| | pH > 1.5 | | |
| | for SiO₂. | | |
| | Does NOT | | |
| | (or less) | | |
| | adsorb to | | |
| | negatively | | |
| | charged | | |
| | surfaces. | | |
| Polyanionic | Adsorbs to | Protein- | Interacts |
| PEG-modified | oxide surfaces | resistant. | specifically |
| copolymers, | with | | with |
| e.g. PLL-g-PEG | positive | | cells (if |
| (see Examples | surface | | ligand = specific |
| 1 and 2), with | charge, | | peptide), |
| part or all of | i.e. at a | | or with DNA |
| the PEG chains | solution pH | | or RNA if |
| functionalized | that is | | ligand is |
| with a bioactive | lower than | | an oligonucleotide |
| ligand | the | | or |
| | isoelectric | | with streptavidin |
| | point of | | if |
| | the oxide | | ligand = biotin. |
| | surface, | | |
| | e.g. at pH > 5 | | |
| | for | | |
| | TiO₂ or at | | |
| | pH > 1.5 | | |
| | for SiO₂. | | |
| | Does NOT | | |
| | (or less) | | |
| | adsorb to | | |
| | negatively | | |
| | charged | | |
| | surfaces. | | |
| Polyanionic or | Adsorbs to | Potein-resistant | Biological |
| polycationic | oxide surfaces | | moiety can |
| PEG-modified | with | | be attached |
| copolymers | positive | | to functional |
| (see Examples | surface | | group |
| 1 and 2), with | charge, | | through covalent |
| part or all of | i.e. at a | | bond, e.g., |
| the PEG chains | solution pH | | peptide, |
| functionalized | that is | | protein, |
| with a reactive | lower than | | enzyme. |
| functional | the | | |
| group | isoelectric | | |
| | point of | | |
| | the oxide | | |
| | surface, | | |
| | e.g. at pH > 5 | | |
| | for | | |
| | TiO₂ or at | | |
| | pH > 1.5 | | |
| | for SiO₂. | | |
| | Does NOT | | |
| | (or less) | | |
| | adsorb to | | |
| | negatively | | |
| | charged | | |
| | surfaces. | | |

After application of the SMAP process, depending on the type of the process and the molecular assembly system used, the surface layer in one of the two (or more) patterns may already contain a biospecific function for interaction with biomolecules or cells or it may contain a suitable reactive (functional) group that allows one to attach biospecific functions. Examples are given in Table. 1

1. Device with chemical surface patterns with biochemical or biological relevance on substrates with prepatterns of at least two different types of regions (α, β, . . . ), whereas at least two different, consecutively applied molecular self-assembly systems (A, B, . . . ) are used in a way that at least one of the applied assembly systems (A or B or . . . ) is specific to one type of the prefabricated patterns (α or β or . . . ).

2. Device according to claim 1 where the specificity is achieved through self-assembly of alkane phosphates or alkane phosphonates from aqueous solutions (assembly system A) in combination with prepatterned surfaces whereas only one type of the prepattern area (α) forms a molecularly assembled layer A of alkane phosphates, while the other prepattern area(s) (β, . . . ) remains uncoated.

3. Device according claim 2 where α is an oxide, nitride or carbide of a metal that chemically interacts with phosphates and/or phosphonates, in particular transition metal oxides such as titanium oxide, tantalum oxide, niobium oxide, zirconium oxide, or non-transition metal oxides that chemically interact with phosphates or phosphonates, and where β is an oxide that does not interact, in particular silicon oxide.

4. Device according to claim 1 where the specificity is achieved through assembly of polyionic, PEG-grafted polymers (B) from aqueous solution at a pH chosen such that one of the two or more prepattern areas (β) is charged oppositely in comparison to the polyionic copolymer and becomes coated by the copolymer due to electrostatic interactions, while the other prepattern area(s) (α) at the same pH carries a charge of same sign as the copolymer and does not or does less become coated.

5. Device according to claim 4 where the prepattern area β is an oxide, nitride or carbide with an isoelectric point (IEP) that is lower than that of area α and the assembly system is a (at the pH of application) polycationic copolymer and the pH of the assembly system solution is chosen between the IEP of area α and area β.

6. Device according to claim 4 where the prepattern area β is an oxide, nitride or carbide with an isoelectric point (IEP) that is higher than that of area α and the assembly system is a (at the pH of application) polyanionic copolymer and the pH of the assembly system solution is chosen between the IEP of area α and area β.

7. Device according to claim 1 where the specificity is achieved through self-assembly of a di- or multiblock copolymer with hydrophobic and hydrophilic segments interacting with a substrate where one of the prepattern area (α) is more hydrophobic than the remaining areas, and therefore gets coated by the di- or multiblock copolymer A while the other prepattern area (β) remains uncoated or less coated.

8. Device according to claim 7 where the di- or multiblock copolymer is a polypropylene oxide (PPO)-poly(ethylene glycol) (PEG) copolymer imparting protein resistance to the more hydrophobic surface.

9. Device according to claim 7 where the hydrophobic prepattern area (α) is composed of a hydrophobic polymer or of an oxide that has been hydrophobized through silanization or application of an alkane phosphate self-assembly system, while the hydrophilic prepattern area is either composed of a hydrophilic polymer or is an inherently hydrophilic oxide or is an oxide that has been made permanently hydrophilic through application of a self-assembled monolayer using a molecule with hydrophilic terminal functional group.

10. Device according to claim 2 where in a second molecular assembly step B the prepattern area β that has not been coated with the alkane phosphate becomes coated with a protein-resistant polymeric layer, leading to a final pattern that is interactive with a biological environment (proteins, cells) in areas A and not interactive (protein- and cell-resistant) in areas B.

11. Device according to claim 10 where B is the assembly of a polyionic PEG coated copolymer, adsorbing onto the oppositely charged area β, e.g. polycationic poly(L-lysine)-g-poly(ethylene oxide) adsorbing at pH of between 2 and 8 onto negatively charged silicon oxide.

12. Device according to claim 4 where in a second step the prepattern area α becomes coated with a functionalized polyionic PEG-grafted copolymer A through application of the second self-assembly solution at a pH different from step 1, at which pH the area α is now oppositely charged in comparison to the polyionic copolymer A and becomes coated with the functionalized polymer, leading to a final pattern that is interactive with a biological environment (proteins, cells) in areas A and non-interactive in areas B.

13. Device according to claim 12 where the polyionic PEG-grated copolymer is functionalized at the end of the PEG chains through covalent linkage to a biologically active group such as biotin interacting with streptavidin, or a peptide or a protein, interacting specifically with receptors in cell membranes.

14. Device according to claim 7 where in a second step the more hydrophilic area (β) that has not been coated in assembly step A gets coated in the second assembly step B with a molecule that induces specific or non-specific interaction with the biological environment.

15. Device according to claim 7 where B is a functionalized polyionic PEG-grafted copolymer according to claim 13 that interacts electrostatically with the oppositely charged surface β or is an alkane phosphate that turns the area β into a hydrophobic, non-specifically interactive area resulting in a final interactive/non-interactive pattern.

16. Device according to claim 13 where a oligo(ethylene oxide) functionalized alkane phosphate is used as the molecular assembly system A, leading to a non-interactive area A, while the area β are subsequently treated with an assembly system B that renders this area interactive, e.g. by adsorbing a functionalized, polyionic PEG-grafted copolymer.

17. Device according to claim 8 where a functionalized (e.g. biotin or peptide or reactive chemical group attached at end of PEG chains) PPO-PEG diblock or PEG-PPO-PEG triblock, or multiblock copolymer is used to render the correspondingly covered area specifically interactive, followed by a second assembly system that renders the remaining area non-interactive, e.g. through adsorption of a polyionic PEG-coated copolymer.

18. Device according to claim 1 where after application of assembly system A and B the resulting interactive/non-interactive pattern is further modified through selective treatment of area A and/or B with biochemically or biologically relevant molecules.

19. Device according to claim 18 where the selective treatment is a nonspecific adsorption of proteins or other biomolecules to the area that is (non-specifically) interactive, e.g. hydrophobic or a selective interaction with ligands previously immobilized in step A or B, e.g. streptavidin interacting specifically with biotin ligand on one of the pattern area.

20. Device according to claim 19 where living cells are added to patterned surfaces and become immobilized selectively on one of the pattern area, through interaction with selectively and nonspecifically adsorbed protein or proteins, or through specific interactions with bioligands such as peptides or proteins that have in a previous step been immobilized through covalent attachment to one of the pattern areas.

21. A bioanalytical sensing platform comprising a device according to claim 1 and at least one biological or biochemical or synthetic recognition element, for the specific recognition and/or binding of one or more analytes and/or for the specific interaction with said analyte(s), immobilized either directly or mediated by a self-assembled layer and/or by an adhesion-promoting layer on at least one of the different types of regions a or b or . . . .

22. A bioanalytical sensing platform according to claim 21, wherein the biological or biochemical or synthetic recognition element is attached to at least one of the applied self-assembly systems A or B, or adsorbs on at least one of said self-assembly systems.

23. A bioanalytical sensing platform according to claim 22, wherein the biological or biochemical or synthetic recognition elements are immobilized in a one-or two-dimensional array of discrete measurement areas, wherein a single discrete measurement area is defined by the area occupied by said immobilized biological or biochemical or synthetic recognition elements on an individual, closed region a or b.

24. A bioanalytical sensing platform according to claim 23, wherein up to 1,000,000 measurement areas are provided in a two-dimensional arrangement on one device with chemical surface pattern, and wherein a single measurement area occupies an area between 10⁻⁴ mm² and 10 mm².

25. A bioanalytical sensing platform according to claim 24, wherein the measurement areas are arranged at a density of at least 10, preferably of at least 100, most preferably of at least 1000 measurement areas per square centimeter.

26. A bioanalytical sensing platform according to claim 25, wherein the biological or biochemical or synthetic recognition elements are selected from the group comprising proteins, such as mono- or polyclonal antibodies or antibody fragments, peptides, enzymes, aptamers, synthetic peptide structures, glycopeptides, oligosaccharides, lectins, antigens for antibodies (e.g. biotin for streptavidin), proteins functionalized with additional binding sites, nucleic acids (such as DNA, RNA, oligonucleotides or polynucleotides) and nucleic acid analogues (such as peptide nucleic acids, PNA) or their derivatives with artificial bases, soluble, membrane-bound proteins, such as membrane-bound receptors and their ligands.

27. A bioanalytical sensing platform according to claim 26, wherein whole cells or cell fragments are immobilized for specific recognition and detection of one or more analytes.

28. A bioanalytical sensing platform according to claim 27, wherein whole cells or cell fragments are immobilized in discrete measurement areas.

29. A bioanalytical sensing platform according to claim 28, wherein less than 100, preferably less than 10, most preferably only 1-3 cells or cell fragments are immobilized per measurement area.

30. A bioanalytical sensing platform according to claim 29, which works for analyte determination by means of a label, which is selected from the group comprising luminescence labels, especially luminescent intercalators or molecular beacons, absorption labels, mass labels, especially metal colloids or plastic beads, spin labels, such as ESR and NMR labels, and radioactive labels.

31. A bioanalytical sensing platform according to claim 29, which is operapable for analyte determination by means of the detection of a change of the effective refractive index in the near field of the surface of said sensing platform due to molecular adsorption on or desorption from said sensing platform.

32. A bioanalytical sensing platform according to claim 29, which is operapable for analyte determination by means of the detection of a change of the conditions for generation of a surface plasmon in a metal layer being part of said sensing platform, wherein said metal layer preferably comprises gold or silver.

33. A bioanalytical sensing platform according to claim 29, which is operapable for analyte determination by means of the detection of a change of one or more luminescences.

34. A bioanalytical sensing platform according to claim 33, which is operapable to receive excitation light in an epi-illumination configuration.

35. A bioanalytical sensing platform according to claim 34, wherein the material of said sensing platform, which is in contact with the measurement areas, is transparent, at least one excitation wavelength, to a depth of at least 200 nm, measured from the surface supporting the immobilized biochemical or biological or synthetic recognition elements in said measurement areas.

36. A bioanalytical sensing platform according to claim 33, which is operapable to receive excitation light in an transmission-illumination configuration.

37. A bioanalytical sensing platform according to claim 36, wherein the materials of said sensing platform are transparent at least one excitation wavelength.

38. A bioanalytical sensing platform according to claim 37, which is operable as an optical waveguide.

39. A bioanalytical sensing platform according to claim 38, characterized in that it is an essentially planar waveguide.

40. A bioanalytical sensing platform according to claim 38, characterized in that it comprises an optically transparent material selected from the group comprising silicates, such as glass or quartz, thermoplastic or moldable plastics, such as polycarbonates, polyimides, acrylates, especially polymethyl methacrylates, and polystyrenes.

41. A bioanalytical sensing platform according to claim 40, characterized in that it comprises an optical thin-film waveguide with a layer (a) being optically transparent at least one excitation wavelength on a layer (b) being optically transparent at least at the same excitation wavelength, wherein the refractive index of layer (b) is lower than the one of layer (a).

42. A bioanalytical sensing platform according to claim 41, wherein the waveguiding layer of said platform is in optical contact to at least one of the optical coupling elements selected from the group comprising prism couplers, evanescent couplers formed by joined optical waveguides with overlapping evanescent fields, distal end (front face) couplers with focusing lenses, preferably cylindrical lenses, located in front of a distal end (front face) of the waveguiding layer, and coupling gratings.

43. A bioanalytical sensing platform according to claim 42, wherein incoupling into the optically transparent layer (a) is performed by means of one or more grating structures (c) formed in layer (a).

44. A bioanalytical sensing platform according to claim 42, wherein outcoupling of light guided in the optically transparent layer (a) is performed by means of one or more grating structures (c′) formed in layer (a), and wherein grating structures (c′) can have the same or different grating period as optional additional grating structures (c).

45. A bioanalytical sensing platform according to claim 44, wherein an array of at least 4 regions with at least two different prefabricated patterns a and b according to claim 1 and, optionally, with one or more self-assembly systems (A, B, . . . ) deposited on the different prefabricated patterns, is located after an incoupling grating (c), with respect to the direction of propagation of light guided in layer (a) after its incoupling by said grating.

46. A bioanalytical sensing platform according to claim 44, wherein an array of at least 4 regions with at least two different prefabricated patterns a and b according to claim 1 and, optionally, with one or more self-assembly systems (A, B, . . . ) deposited on the different prefabricated patterns, is located on a coupling grating (c) or (c′).

47. A bioanalytical sensing platform according to claim 46, wherein a continuous coupling grating (c) or (c′) extends over at least 30% of the surface of said sensing platform.

48. A bioanalytical sensing platform according to claim 47, wherein an additional, at least at one excitation wavelength optically transparent, layer (b′) with lower refractive index than and in contact with layer (a), and with a thickness of 5 nm-10 000 nm, preferably of 10 nm-1000 nm, is located between the optically between the optically transparent layers (a) and (b).

49. A bioanalytical sensing platform according to claim 48, wherein layer (b) comprises an optically transparent (i.e. optically transparent at least one excitation wavelength) material selected from the group comprising silicates, such as glass or quartz, thermoplastic or moldable plastics, such as polycarbonates, polyimides, acrylates, especially polymethyl methacrylates, and polystyrenes.

50. A bioanalytical sensing platform according to claim 49, wherein the refractive index of layer (a) is higher than 1.8.

51. A bioanalytical sensing platform according to claim 50, wherein layer (a) comprises a material selected from the group comprising TiO₂, ZnO, Ta₂O₅, HfO₂, and ZrO₂, preferably especially from the group comprising TiO₂, Ta₂O₅, and Nb₂O₅.

52. A bioanalytical sensing platform according to claim 51, wherein the thickness of layer (a) is between 40 and 300 nm, preferably between 70 and 200 nm.

53. A bioanalytical sensing platform according to claim 52, wherein gratings (c) or (c′) have a period of 200 nm-1000 nm and a modulation depth of 3 nm-100 nm, preferably of 10 nm-30 nm.

54. A method for the simultaneous qualitative and/or quantitative determination of one or more analytes in one or more samples, wherein said samples are brought into contact with the measurement areas on a bioanalytical sensing platform according to claim 21, and wherein the resulting changes of signals from said measurement areas are measured.

55. A method according to claim 54, wherein said changes of signals from the measurement areas are obtained upon using a label, which is selected from the group comprising luminescence labels, especially luminescent intercalators or molecular beacons, absorption labels, mass labels, especially metal colloids or plastic beads, spin labels, such as ESR and NMR labels, and radioactive labels.

56. A method according to claim 54, wherein analyte determination is performed upon detection of a change of the effective refractive index in the near field of the surface of said sensing platform due to molecular adsorption on or desorption from said sensing platform.

57. A method according to claim 54, wherein analyte determination is performed upon detection of a change of the conditions for generation of a surface plasmon in a metal layer being part of said sensing platform, wherein said metal layer preferably comprises gold or silver.

58. A method according to claim 54, wherein analyte determination is performed upon detection of a change of one or more luminescences.

59. A method according to claim 58, wherein excitation light from one or more light sources is launched on the bioanalytical sensing platform in a configuration of epi-illumination.

60. A method according to claim 58, wherein excitation light from one or more light sources is launched on the bioanalytical sensing platform in a configuration of transmission-illumination.

61. A method according to claim 58, wherein the bioanalytical sensing platform comprises an optical waveguide, which is preferably essentially planar, and wherein excitation light from one or more light sources is coupled into said waveguide by means of an optical coupling element selected from the group comprising prism couplers, evanescent couplers formed by joined optical waveguides with overlapping evanescent fields, distal end (front face) couplers with focusing lenses, preferably cylindrical lenses, located in front of a distal end (front face) of the waveguiding layer, and coupling gratings.

62. A method according to claim 61, wherein said bioanalytical sensing platform comprises an optical thin-film waveguide, with a first optically transparent layer (a) on a second optically transparent layer (b) with lower refractive index than layer (a), wherein furthermore excitation light is incoupled into the optically transparent layer (a) by one or more grating structures formed in the optically transparent layer (a), and directed, as a guided wave, to the measurement areas located thereon, and wherein furthermore the luminescence from molecules capable to luminesce, which is generated in the evanescent field of said guided wave, is detected by one or more detectors, and wherein the concentration of one or more analytes is determined from the intensity of these luminescence signals.

63. A method according to claim 62, wherein (1) the isotropically emitted luminescence or (2) luminescence that is incoupled into the optically transparent layer (a) and outcoupled by a grating structure (c) or (c′) or luminescence comprising both parts (1) and (2) is measured simultaneously.

64. A method according to claim 63, wherein, for the generation of said luminescence, a luminescent dye or a luminescent nano-particle is used as a luminescence label, which can be excited and emits at a wavelength between 300 nm and 1100 nm.

65. A method according to claim 64, for the simultaneous or sequential, quantitative or qualitative determination of one or more analytes of the group comprising wherein antibodies or antigens, receptors or ligands, chelators or histidin-tag components, oligonucleotides, DNA or RNA strands, DNA or RNA analogues, enzymes, enzyme cofactors or inhibitors, lectins and carbohydrates.

66. A method according to claim 64, wherein the samples to be examined are naturally occurring body fluids, such as blood, serum, plasma, lymphe or urine or egg yolk or optically turbid liquids or surface water or soil or plant extracts or bio- or process broths or are taken from biological tissue.

67. The use of a bioanalytical sensing platform according to claim 21 and/or of a method according to claim 54 for quantitative or qualitative analysis for the determination of chemical, biochemical or biological analytes in screening methods in pharmaceutical research, combinatorial chemistry, clinical and preclinical development, for real-time binding studies and the determination of kinetic parameters in affinity screening and in research, for qualitative and quantitative analyte determinations, especially for DNA- and RNA analytics, for the generation of toxicity studies and the determination of expression profiles and for the determination of antibodies, antigens, pathogens or bacteria in pharmaceutical product development and research, human and veterinary diagnostics, agrochemical product development and research, for patient stratification in pharmaceutical product development and for the therapeutic drug selection, for the determination of pathogens, nocuous agents and germs, especially of salmonella, prions and bacteria, in food and environmental analytics.

68. Device according to claim 1 comprising a biomedical device with patterns in the size range of cells, typically 5 to 100 micrometer, interconnected or not, isotropic or anisotropic, to influence or control cell form and attachment area, cell morphology, cytoskeleton organization, cell proliferation, cell differentiation and the expression of factors within the cell and to the extracellular matrix.

69. A biomedical device according to claim 68, where cells are osteogeneic precursor cells, osteoblasts, osteoclasts, fibroblasts, smooth muscle cells, endothelial cells, epithelial cells, nerve cells, macrophages.

70. Device according to claim 1 comprising a biomedical device with patterns of size below 5 micrometer and above 10 nanometer, which are representative of subcellular features such as membrane receptors or focal contacts in order to influence the formation of stress fibres, the organization of the cytoskeleton and the migration of the cell at the surface.

71. A biomedical device fabricated according to claims 68 or 70 with cell-adhesive patterns that contain specific ligands such as peptides, proteins and antibodies and that are used to interact more specifically with one kind of cells than with others with the aim to influence the formation of assembly of preferred cell types and the formation of a preferred type of tissue at the implant/body interface.

72. A biomedical device fabricated according to claims 68 or 70 with cell-adhesive patterns that contain specific ligands such as peptides and that are used to interact specifically with one or a selected number of cell membrane receptors, e.g. of the integrin receptor or heparin-type receptor type.

73. Patterns according to claim 71, whereby the peptides contain one or several of the following amino acid sequences: RGD, KRSR, YIGSG, FHRRIKA, DGEA, CSRARKQAASIKVAVSADR, MAPLRPLLIL, ALLAWVALAD, QESCKGRCTE, GFNVDKKCQC, DELCSYYQSC, CTDYTAECKP, QVTRGDVFTM, PEDEYTVYDD, GEEKNNATVH, EQVGGPSLTS, DLQAQSKGNP, EQTPVLKPEE, EAPAPEVGAS, KPEGIDSRPE, TLHPGRPQPP, AEEELCSGKP, FDAFTDLKNG, SLFAFRGQYC, YELDEKAVRP, GYPKLIRDVW, GIEGPIDAAF, TRINCQGKTY, LFKGSQYWRF, EDGVLDPDYP, RNISDGFDGI, PDNVDAALAL, PAHSYSGRER, VYFFKGKQYW, EYQFQHQPSQ, EECEGSSLSA, VFEHFAMMQR, DSWEDIFELL, FWGRTSAGTR, QPQFISRDWH, GVPGQVDAAM, AGRIYISGMA, PRPSLAKKQR, FRHRNRKGYR, SQRGHSRGRN, QNSRRPSRAT WLSLFSSEES, NLGANNYDDY, RMDWLVPATC, EPIQSVFFFS, GDKYYRVNLR, TRRVDTVDPP, YPRSIAQYWL, GCPAPGHL, MRIAVICFCL, LGITCAIPVK, QADSGSSEEK, QLYNKYPDAV, ATWLNPDPSQ, KQNLLAPQTL, PSKSNESHDH, MDDMDDEDDD, DHVDSQDSID, SNDSDDVDDT, DDSHQSDESH, HSDESDELVT, DFPTDLPATE, VFTPVVPTVD, TYDGRGDSVV, YGLRSKSKKF, RRPDIQYPDA, TDEDITSHME, SEELNGAYKA, IPVAQDLNAP, SDWDSRGKDS, YETSQLDDQS, AETHSHKQSR, LYKRKANDES, NEHSDVIDSQ, ELSKVSREFH, SHEFHSHEDM, LVVDPKSKEE, DKHLKFRISH, ELDSASSEVN, MKTALILLSI, LGMACAFSMK, NLHRRVKIED, SEENGVFKYR, PRYYLYKHAY, FYPHLKRFPV, QGSSDSSEEN, GDDSSEEEEE, EEETSNEGEN, NEESNEDEDS, EAENTTLSAT, TLGYGEDATP, GTGYTGLAAI, QLPKKAGDIT, NKATKEKESD, EEEEEEEEGN, ENEESEAEVD, ENEQGINGTS, TNSTEAENGN, GSSGGDNGEE, GEEESVTGAN, AEGTTETGGQ, GKGTSKTTTS, PNGGFEPTTP, PQVYRTTSPP, FGKTTTVEYE, GEYEYTGVNE, YDNGYEIYES, ENGEPRGDNY, RAYEDEYSYF, KGQGYDGYDG, QNYYHHQ, STGSKQRSQN, RSKTPKNQEA, SNVILKKRYN, MVVRACQCH.

74. Biomedical device according to claim 68, where the size of the adhesive sites are chosen such that macrophages can adsorb to such patterns, but not nucleate into polynuclear cells of the Foreign Body Giant Cell (FBGC) type.

75. Biomedical device according to claim 68 where the patterns are applied to three-dimensional objects.

76. Biomedical device according to claim 75, where the objects are products or components of products such as catheters, stents, dental and maxillofacial implants, osteosynthesis plates or screws, artificial joint components, spine surgery device such as cages, vascular and cardiovascular devices such as heart valves or audiological devices, all of which are used in contact with a biological environment in a living body (“in vivo”), such as body fluid, blood, biological tissue.

77. Biomedical device according to claim 68, which is used as a substrate in cell culture testing (“in vitro”) to influence and organize cell attachment to such substrate.

78. Biomedical device according to claim 68, whereby the substrate is made out of a metal or alloy, a polymer, a ceramic material or a composite material.

79. Endoprosthesis and implant according to claim 78 used in joint replacement (hip, knee, ankle, shoulder, elbow, wrist, finger, etc.) and bone fracture fixation (plates, screws, pins, nails, etc.) respectively where their whole or selected parts of their contact surface area with hard or soft tissue respectively is patterned by SMAP. These endoprostheses and implants, i.e. the substrate for application of SMAP may consist of metal, polymer or ceramic materials as well as of combinations of these materials types, i.e. composites. Such endoprostheses and implants are intended to be used in humans and animals, alone or in combination with any additional auxiliary materials like bone cement, bioactive or bioinductive substances.
